(19) United States
(12) Patent Application Publication
(10) Pub. No.: US 2021/0371852 A1
(43) Pub. Date: Dec. 2, 2021

(54) SYSTEM OF CELL EXPANSION AND METHODS OF USING THE SAME

(71) Applicant: CHILDREN'S NATIONAL MEDICAL CENTER, Washington, DC (US)

(72) Inventors: Catherine Mary Bollard, Bethesda, MD (US); Conrad Russell Y. Cruz, Bethesda, MD (US); Patrick Hanley, Washington, DC (US)

(73) Assignee: CHILDREN'S NATIONAL MEDICAL CENTER, Washington, DC (US)

(21) Appl. No.: 17/056,710

(22) PCT Filed: May 20, 2019
(86) PCT No.: PCT/US2019/033187

§ 371 Date: Nov. 18, 2020

Related U.S. Application Data
(60) Provisional application No. 62/673,810, filed on May 18, 2018.

(51) Int. Cl. C12N 15/10; C12M 1/12; C12N 5/0783; C12M 1/00

(52) U.S. Cl. CPC C12N 15/1093; C12M 29/16; C12N 5/0636; C12M 25/10

(57) ABSTRACT

The present disclosure relates, at least in part, to a closed and semi-automated system for the isolation of naive T cells, their expansion, and/or final harvest. The disclosure also relates to using those isolated cells in a large batch format for compiling stocks of stimulated CD45A+ T cells and/or using the stimulated CD45A+ T cells for therapeutic purposes.

### CROSS-REFERENCE TO RELATED APPLICATION

This application claims the benefit of provisional U.S. Application No. 62/673,810, filed, May 18, 2018, the entirety of which is hereby incorporated by reference for all purposes.

### FIELD OF INVENTION

The present invention relates generally to novel methods of generating, culturing and expanding antigen-specific T-cells.

### BACKGROUND

Current Good Manufacturing Practices (cGMP) are required by the Food and Drug Administration (FDA) for all drugs manufactured for patients. The introduction of biologics, however, has challenged the former paradigm of GMP, requiring personalized medicines that are often manufactured individually for each patient which makes adherence to full GMP challenging. In addition, the use of living cells, which are highly variable, introduces uncertainty into the process which makes designing potency assays difficult and requires that SOPs be broad. Beyond these challenges, cellular therapies currently lack the technology to manufacture products that can be grown on a small scale for each patient in a closed fashion, where there is limited-to-no exposure to the environment. Moreover there is an unmet need to create processes that enable the closed manufacturing of antigen-specific T cells, especially in an automated or semi-automated way.

### SUMMARY OF THE DISCLOSURE

The disclosure relates, at least in part, to the isolation of naïve cells, their expansion, and final harvest in a closed and semi-automated system. As described by the present disclosure, during the manufacture of T cells specific for Tumor Associated Antigens (TAA) or virus-specific T cells where the donor is seronegative (such as cord blood or adult seronegative donors), the expanded T cell product will be derived from the naïve T cell population instead of the memory T cell population, which has been the source of T cells in many other cellular therapy protocols. Thus, by selecting for the naïve T cell population, the population that will respond to stimulation will be enriched, leading to a superior expansion and final therapeutic product. The methods described by the present disclosure advantageously provide large scale production of the final therapeutic product.

In one aspect, the disclosure features a system comprising a cell culture unit comprising one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet; a gas transfer module in operable connection to the one or plurality of cell reactor surfaces; and a first gas inlet in operable connection to the gas transfer module.

In one embodiment, the one or plurality of cell reactor surfaces have a surface area from about 2 meters squared to about 100 meters squared. In one embodiment, the one or plurality of cell reactor surfaces are configured in a cylindrical form with a hollow volume fixed within a cylindrical first compartment; wherein the first media line and the second media line are positioned on opposite faces of the cylinder. In one embodiment of the above aspects and embodiments, the first media line is attached to a first sealable aperture configured for sterile attachment of a cell culture media source. In one embodiment of the above aspects and embodiments, the first gas inlet is attached to a second sealable aperture configured for sterile attachment of a gas source. In one embodiment of the above aspects and embodiments, the system further comprises an apheresis unit in fluid communication with the cell culture unit. In one embodiment of the above aspects and embodiments, the system further comprise a harvesting compartment in fluid communication with the cell culture unit. In one embodiment of the above aspects and embodiments, the system further comprises a pump and a fluid regulator in operable contact with the first media line, wherein the pump is capable of generating pressure in the first media line and wherein the fluid regulator is capable of regulating the speed of fluid from the pump through the first compartment and into the second media line. In one embodiment of the above aspects and embodiments, the gas module comprises a gas pump and a gas regulator connected to the first compartment by a first gas line; wherein the first compartment comprises at least one gas outlet; wherein the gas pump is capable of generating air pressure from the pump to the first compartment through the first gas line, wherein the at least one gas outlet is a vent or in configured for sterile connection to a vent; and wherein the gas regulator is capable of regulating the speed of gas from the pump through the first compartment. In one embodiment of the above aspects and embodiments, the system further comprises one or a plurality of CD45a+ T-cells from a subject. In one embodiment of the above aspects and embodiments, the system further comprises one or a plurality of dendritic cells from a subject.

In another aspect, the disclosure features a method of expanding CD45A+ T-cells from a subject comprising (a) culturing one or a plurality of CD45A+ T-cells in the system of any one of disclosed embodiments; and (b) allowing the CD45A+ T-cells to grow in the first compartment for a time period sufficient to proliferate. In one embodiment, the method further comprises introducing CD45A+ T-cells into the first compartment of the system of any of the aspects and embodiments herein. In one embodiment of the above aspects and embodiments, the CD45A+ T-cells are allowed to grow for a time period sufficient to proliferate into a total cell number of from about 1×10⁹ to about 1×10¹² cells. In one embodiment, the step of culturing comprises co-culturing the CD45A+ T-cells with one or a plurality of dendritic cells. In one embodiment of the above aspects and embodiments, the method further comprises a step of allowing one or plurality of dendritic cells presenting at least one antigen to contact one or plurality of CD45A+ T-cells for a period of time sufficient to stimulate a T-cell response against the at least one antigen. In one embodiment of the above aspects and embodiments, the dendritic cells and the CD45A+ T-cells are from a subject.

[01] The disclosure also relates to a method of harvesting stimulated T cell populations derived from healthy subjects, the method comprising: (a) isolating CD45A+ T cells from a subject; (b) culturing one or a plurality of CD45A+ T-cells in the system of any one of disclosed embodiments; and, concurrently, (c) isolating antigen presenting cells, such as dendritic cells from a sample; (d) exposing the antigen presenting cells to one or a plurality of any antigens disclosed herein for a time period sufficient for the antigen presenting cells to process the one or plurality of antigens; (e) co-culturing the dendritic cells with the isolated CD45A+ T-cells in a cell culture unit; (f) optionally exposing the CD45A+ T cells to one or a plurality of immunostimulatory molecules, such as cytokines and chemokines disclosed herein, for a time period sufficient to stimulate an antigen-specific response with the CD45A+ T cells; and (g) harvesting CD45A+ T cells. In some embodiments, the methods further comprise allowing CD45A+ T cells to grow and/or expand in culture for from about 3-9 days before the step of harvesting. In some embodiments, the steps of isolating the antigen presenting cells and/or the CD45A+ T cells comprises performing apheresis. In some embodiments, the antigen presenting cells are dendritic cells. In some embodiments, the antigen presenting cells are exposed to WT1, PRAME and/or surviving sequentially or contemporaneously for a time period sufficient for the antigen presenting cells to process the one or plurality of antigens. In some embodiments, the methods of culturing or isolating or harvesting CD45A+ T cells comprises exposing the antigen presenting cells or the CD45A+ T cells to a flow cytometry step and then introducing the cells to other cell population adherent in the cell culture unit for a time period sufficient to stimulate an antigen-specific response in the CD45A+ T cells. In some embodiments, the CD45A+ T cells are derived from a healthy subject or a subject free of cancer. In some embodiments, the CD45A+ T cells are derived from a sample of serum. In some embodiments, any of the methods disclosed herein further comprise a step of obtaining a sample of blood from a subject.

In another aspect, the present disclosure features a method of isolating antigen-stimulated CD45A+ T-cells comprising (a) culturing one or a plurality of CD45A+ T-cells in the system of any one of the aspects and embodiments herein; (b) allowing the CD45A+ T-cells to grow in the first compartment for a time period sufficient to proliferate; (c) allowing one or plurality of dendritic cells presenting at least one antigen to contact one or plurality of CD45A+ T-cells for a period of time sufficient to stimulate a T-cell response against the at least one antigen; and (d) harvesting the one or plurality of CD45A+ T-cells in a closed system.

### BRIEF DESCRIPTION OF THE DRAWINGS

FIG. 1 depicts a flow chart of a culturing and harvesting method.

FIG. 2 depicts the Quantum bioreactor (TerumoBCT). The bioreactor is constructed of 10,000 hollow fibers that total about 2.1 meters squared of surface area. Inside the hollow fibers is the intracapillary space where cells are fed with media. Between hollowfibers is the extracapillary space which has many functions, including ultrafiltration where we can feed from the EC sign at a rate that causes non-MSC cells to detach and wash away into the waste bag.

FIG. 3 depicts the interior portion of the Quantum: the expansion set. In the top right corner are the inlet and outlet lines, that include lines for reagents such as TrypLE select, a wash line for PBS, a cell line, and and a harvest line. There is also an IC media line for feeding cells from the intracapillary space and an EC media line for feeding from the extra capillary space.

FIG. 4 depicts a schematic of the touch-screen interface. There are two media lines, IC and EC. Each has its own designated inlet rate and circulation rate. Depending on the growth of the cells, we can adjust the inlet rate to feed the cells more often. One advantage of having the IC line and the EC line is that two media bags cab be hooked up, and a task can be set up that will change the feeding of one bag to the other bag once the first bag is empty.

FIG. 5 depicts a closed system for the culturing, stimulation and/or harvesting of antigen-primed T cells.

FIG. 6 depicts a flowchart of the an alternative embodiment of isolation and culturing of lymphocytes in which dendritic cells are stimulated with antigen and are co-cultured with T-cell populations.

FIG. 7 depicts a schematic of a tissue culture system comprising a plurality of compartments separated by a partition, wherein each compartment has a defined surface area of cell reactor surface. The cell reactor surface may be coated with one or a series of molecules or modifications such as antibodies and/or cyclodextrin or the reactor surface may be free of any one or multiple modifications such as antibodies or cyclodxtrin. When cells reach a certain density within the system, FIG. 7 depicts that one or a plurality of partitions may be removed such that additional surface area is exposed for T-cells to proliferate into the additional surface area of the compartments.

### DETAILED DESCRIPTION OF EMBODIMENTS

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

### Definitions

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues (e.g. tag and targeting peptides as mentioned herein). “Consisting essentially of” means excluding other components or steps of any essential significance. Thus, a composition consisting essentially of the recited components would not exclude trace contaminants and pharmaceutically acceptable carriers. A polypeptide “consists essentially of an amino acid sequence when such an amino acid sequence is present with eventually only a few additional amino acid residues. “Consisting of means excluding more than trace elements of other components or steps. For example, a polypeptide “consists of an amino acid sequence when the polypeptide does not contain any amino acids but the recited amino acid sequence.

As used herein, “substantially equal” means within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

The term “allogeneic” as used herein refers to medical therapy in which the donor and recipient are different individuals of the same species. In some embodiments, the donor and recipient are HLA matched.

As used herein, the term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, including but not limited to peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

As used herein, the term “antigen presenting cell (APC)” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

As used herein, the term “autologous” as used herein refers to a medical therapy in which the donor and recipient are the same subject.

As used herein, “cell culture” means growth, maintenance, transfection, transduction and/or propagation of cells, tissues, or their products. As used herein, “culture medium” refers to any solution or suspension capable of sustaining the growth of the targeted cells either in vitro or in vivo, or any solution with which targeted cells or exogenous nucleic acids are mixed before being applied to cells in vitro or to a patient in vivo.

As used herein, the term “cord blood” as used herein has its normal meaning in the art and refers to blood that remains in the placenta and umbilical cord after birth and contains hematopoietic stem cells. Cord blood may be fresh, cryopreserved, or obtained from a cord blood bank.

As used herein, the term “cytokine” as used herein has its normal meaning in the art. Nonlimiting examples of cytokines used in the invention include IL-2, IL-6, IL-7, IL-12, IL-15, and IL-21.

As used herein, the term “cytotoxic T-cell” or “cytotoxic T lymphocyte” is a type of immune cell that bears a CD8+ antigen and that can kill certain cells, including foreign cells, tumor cells, and cells infected with a virus. Cytotoxic T cells can be separated from other blood cells, grown ex vivo, and then given to a patient to kill tumor or viral cells. A cytotoxic T cell is a type of white blood cell and a type of lymphocyte.

As used herein, the term “dendritic cell” or “DC” describes a diverse population of morphologically similar cell types found in a variety of lymphoid and non-lymphoid tissues, see Steinman, Ann. Rev. Immunol. 9:271-296 (1991).

As used herein, the term “therapeutically effective amount” means a quantity sufficient to achieve a desired therapeutic or prophylactic effect, for example, an amount which results in the prevention or amelioration of or a decrease in the symptoms associated with a disease that is being treated, e.g., cancer. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. The compound of the invention can be administered to the subject either prior to or after the onset of a disease or disorder, for example cancer. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation. Typically, an effective amount of the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds. Generally, therapeutically effective amount refers to an amount of a composition or pharmaceutical composition that ameliorates symptoms, or reverses, prevents or reduces the rate of progress of disease, or extends life span of an individual when administered alone or in combination with other therapeutic agents or treatments as compared to the symptoms, rate of progress of disease, or life span of an individual not receiving a therapeutically effective amount an inhibitor disclosed herein.

The term “endogenous” as used herein refers to any material from or produced by an organism, cell, tissue or system. In some embodiments, the material from or produced by an organism, cell, tissue or system is free of any external components such as recombinant nucleic acid introduced into the organism, cell, tissue or system.

As used herein, the term “epitope” corresponds to a minimal peptide motif (usually a set of 8-25 amino acid residues) that forms a site recognized by an antibody, a T-cell receptor or a HLA molecule. Those residues can be consecutive (linear epitope) or not (conformational epitope that includes residues that are not immediately adjacent to one another).

As used herein, the term “exogenous” refers to any material introduced from or produced outside an organism, cell, tissue or system or by non-naturally occurring material introduced into the organism, cell, tissue or system.

As used herein, the term “HLA” refers to human leukocyte antigen. There are 7,196 HLA alleles. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8-positive (i.e., cytotoxic) T-cells. HLAs corresponding to MHC Class II (DP, DM, DOA, DOB, DQ and DR) stimulate the multiplication of CD4-positive T-cells) which stimulate antibody-producing B-cells.

As used herein, the term “immunogenic” refers to the ability to induce or stimulate a measurable T and/or B cell-mediated immune response in a subject into which the component qualified as immunogenic has been introduced. For example, the antigenic combination of the invention is immunogenic in the sense as it is capable of inducing or stimulating an immune response in a subject or within one or a plurality of disclosed cells which can be innate and/or specific (i.e. against at least one cancer antigen/epitope comprised in or expressed by said immunogenic combination), humoral and/or cellular (e.g. production of antibodies and/or cytokines and/or the activation of cytotoxic T cells, B, T lymphocytes, antigen presenting cells, helper T cells, dendritic cells, NK cells, etc) and usually results in a protective response in the administered subject. A vast variety of direct or indirect biological assays are available in the art to evaluate the immunogenic nature of a component either in vivo (animal or human being), or in vitro (e.g. in a biological sample) as described herein.

As used herein, the term “immune effector cell,” as that term is used herein, refers to a cell that is involved in an immune response, e.g., in the promotion of an immune effector response. Examples of immune effector cells include T cells, (e.g., alpha/beta T cells and gamma/delta T cells, B cells, natural killer (NK) cells, natural killer T (NKT) cells, mast cells, and myeloic-derived phagocytes).

As used herein, the term “immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

As used herein, the phrase “in need thereof” means that the subject or mammal has been identified or suspected as having a need for the particular method or treatment. In some embodiments, the identification can be by any means of diagnosis or observation. In any of the methods and treatments described herein, the subject or mammal can be in need thereof. In some embodiments, the animal or mammal is in an environment or will be traveling to an environment in which a particular disorder or condition is prevalent or more likely to occur.

As used herein, a “naive” T-cell or other immune effector cell is meant to refer to one that has not been exposed to or primed by an antigen or to an antigen-presenting cell presenting a peptide antigen capable of activating that cell.

“Antigen specific T cell” as used herein is intended to refer to T cells that recognise a particular antigen and responds thereto, for example by proliferating and/or producing cytokines in response thereto.

As used herein the term “passaging” is meant to refer to a technique that enables cells to be kept alive and growing under cultured conditions for extended periods of time. Passaging involves transferring some or all cells from a previous culture to fresh growth medium. Cells are generally passaged when they reach confluence.

As used herein, a “peptide library” or “overlapping peptide library” is meant to refer to a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries are commercially available and may be custom-made for particular antigens. Methods for contacting, pulsing or loading antigen-presenting cells are well known and incorporated by reference to Ngo, et al (2014), Peptide libraries may be obtained from JPT and are incorporated by reference to the website at https://www.jpt.com/products/peptrack/peptide-libraries.

As used herein, a “peripheral blood mononuclear cell” or “PBMC” refers to peripheral blood cell having a round nucleus. These cells consist of lymphocytes (T cells, B cells, NK cells) and monocytes. In humans, lymphocytes make up the majority of the PBMC population, followed by monocytes, and only a small percentage of antigen presenting cells, such as dendritic cells.

As used herein, the term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

As used herein, a “T-cell population” or “T-cell subpopulation” is intended to include thymocytes, immature T lymphocytes, mature T lymphocytes, resting T lymphocytes and activated T-lymphocytes. The T-cell population or subpopulation can include αβ T-cells, including CD4+ T-cells, CD8+ T cells, γδ T-cells, Natural Killer T-cells, or any other subset of T-cells.

As used herein, the term “subject” is used throughout the specification to describe an animal from which a cell sample is taken. In some embodiments, the subject is a human. For diagnosis of those conditions which are specific for a specific subject, such as a human being, the term “patient” may be interchangeably used. In some instances in the description of the present invention, the term “patient” will refer to human patients suffering from a particular disease or disorder. In some embodiments, the subject is an animal, such as a mammal. As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal. As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human. In some embodiments, the mammal refers to any non-human mammal. The present disclosure relates to any of the methods or compositions of matter disclosed herein wherein the sample is taken from a mammal or non-human mammal. The present disclosure relates to any of the methods or compositions of matter disclosed herein wherein the sample is taken from a human or non-human primate.

As used herein, the term “tumor-associated antigen expression profile” or “tumor antigen expression profile” as used herein, refers to a profile of expression levels of tumor-associated antigens within a malignancy or tumor. Tumor-associated antigen expression may be assessed by any suitable method known in the art including, without limitation, quantitative real time polymerase chain reaction (qPCR), cell staining, or other suitable techniques. Non-limiting exemplary methods for determining a tumor-associated antigen expression profile can be found in Ding et al., Cancer Bio Med (2012) 9: 73-76; Qin et al., Leukemia Research (2009) 33(3) 384-390; Weber et al., Leukemia (2009) 23: 1634-1642; Liu et al., J. Immunol (2006) 176: 3374-3382; Schuster et al., Int J Cancer (2004) 108: 219-227.

As used herein, the terms “tumor-associated antigen” or “TAA” as used herein is an antigen that is highly correlated with certain tumor cells. They are not usually found, or are found to a lesser extent, on normal cells.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

The phrase “pharmaceutically acceptable” refers to molecular entities and compositions that are physiologically tolerable and do not typically produce an allergic or similar untoward reaction, such as gastric upset, dizziness and the like, when administered to a subject, such as a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which cannot be considered pharmaceutically acceptable, can be useful in the preparation of salts useful as intermediates in obtaining a chemical compound of the invention and its pharmaceutically acceptable acid addition salt. Metal salts of a chemical compound of the invention include alkali metal salts, such as the sodium salt of a chemical compound of the invention containing a carboxy group. Mixtures of isomers obtainable according to the invention can be separated in a manner known per se into the individual isomers; diastereoisomers can be separated, for example, by partitioning between polyphasic solvent mixtures, recrystallization and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

As used herein, “control sample” or “reference sample” refer to samples with a known presence, absence, or quantity of substance being measured, that is used for comparison against an experimental sample.

The present disclosure describes methods that have significant advantages for isolating and expanding naïve T cells targeting TAAs. The methods described herein minimize the amount of time, intervention and resource required to produce a therapeutic product.

### Cell Culture System

In some embodiments, a system comprising a cell culture unit is utilized to culture and expand a T-cell population described herein. In some embodiments, the cell culture unit comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments, the one or plurality of cell reactor surfaces are configured in a cylindrical form with a hollow volume fixed within a cylindrical first compartment; wherein the first media line and the second media line are positioned on opposite faces of the cylindrical first compartment. The first media line can be attached to a first sealable aperture configured for sterile attachment of a cell culture media source. In some embodiments, the system further comprises a pump and a fluid regulator in operable contact with the first media line, wherein the pump is capable of generating pressure in the first media line and wherein the fluid regulator is capable of regulating the speed of fluid from the pump through the first compartment and into the second media line.

The one or plurality of cell reactor surfaces can have a surface area from about 0.5 m² to about 100.0 m², including any value therein, such as about 3 m², about 4 m², about 5 m², about 6 m², about 7 m², about 8 m², about 9 m², about 10 m², about 11 m², about 12 m², about 13 m², about 14 m², about 15 m², about 16 m², about 17 m², about 18 m², about 19 m², about 20 m², about 21 m², about 22 m², about 23 m², about 24 m², about 25 m², about 26 m², about 27 m², about 28 m², about 29 m², about 30 m², about 31 m², about 32 m², about 33 m², about 34 m², about 35 m², about 36 m², about 37 m², about 38 m², about 39 m², about 40 m², about 41 m², about 42 m², about 43 m², about 44 m², about 45 m², about 46 m², about 47 m², about 48 m², about 49 m², about 50 m², about 51 m², about 52 m², about 53 m², about 54 m², about 55 m², about 56 m², about 57 m², about 58 m², about 59 m², about 60 m², about 61 m², about 62 m², about 63 m², about 64 m², about 65 m², about 66 m², about 67 m², about 68 m², about 69 m², about 70 m², about 71 m², about 72 m², about 73 m², about 74 m², about 75 m², about 76 m², about 77 m², about 78 m², about 79 m², about 80 m², about 81 m², about 82 m², about 83 m², about 84 m², about 85 m², about 86 m², about 87 m², about 88 m², about 89 m², about 90 m², about 91 m², about 92 m², about 93 m², about 94 m², about 95 m², about 96 m², about 97 m², about 98 m², or about 99 m², or about 100 m², or about 105 m².

The system further comprises a gas transfer module in operable connection to the one or plurality of cell reactor surfaces. In some embodiments, the gas module comprises a gas pump and a gas regulator connected to the first compartment by a first gas line. In such embodiments, the first compartment comprises at least one gas outlet. The gas pump is capable of generating air pressure from the pump to the first compartment through the first gas line. The gas outlet can be one or more vents or the gas outlet can be configured for sterile connection to one or more vents. The gas regulator is capable of regulating the speed of gas from the pump through the first compartment.

Some embodiments further comprise a first gas inlet in operable connection to the gas transfer module. In some embodiments, the first gas inlet is attached to a second sealable aperture configured for sterile attachment of a gas source. The gas source can be any known gas storage and/or delivery system, such as for example a container or a tank.

The system can further comprise an apheresis unit in fluid communication with the cell culture unit. Suitable apheresis units include the Spectra Optia Apheresis System (TerumoBCT).

Additionally, in some embodiments, the system further comprises a harvesting compartment in fluid communication with the cell culture unit. Suitable harvesting compartments are discussed elsewhere herein.

A cell culture system as described herein can be used to expand T-cells from a subject through culturing one or a plurality of T-cells in the system and allowing the T-cells to grown in the first compartment for a time period sufficient to proliferate. The T-cells can be introduced into the system through the system's first compartment. In some embodiments, the T-cells are CD45A+ T-cells.

The disclosure also relates to a system comprising a cell culture unit comprising one or a plurality of cell reactor surfaces housed in a plurality of compartments, each compartment separated by a removable partition first compartment comprising at least one cell reactor surface, at least one cell reactor surface in fluid connection with a first and second media line, the first media line in fluid communication with a first media inlet, the second media line in fluid communication to a first media outlet. In some embodiments, the cell culture unit comprises a single cell culture chamber comprising multiple partitions, each partition independently removable and independently in fluid connection with the first and the second media line and each partition or set of partitions defining a distinct compartment. In some embodiments, the cell culture unity comprises 2, 3, 4, 5, 6, 7, 8, 9, 10, 11, 12 or more compartments, each compartment separated by and/or defined by one or more partitions. In some embodiments, the compartments are configured in a grid or linear pattern. In some embodiments, each partition separating one compartment from another compartment may be removed such that the cell reactor surface of a first compartment is or becomes contiguous with a cell reactor surface of a second compartment. The removal of one or more partitions allows for an increased surface area onto which cells from one compartment (such as the first compartment) may proliferate and/or grow into another compartment (such as the second compartment) during a method of culturing. In some embodiments, the cell culture unit comprises a set of side walls defining a single surface area divided among 2, 3, 4, 5, 6, 7, 8, 9, 10, 11, 12 or more compartments each compartment with at least one or a plurality of cell reactor surfaces. In some embodiments, each compartment has at least a first cell reactor surface. The disclosure relates to a method of growing T-cell populations on a tissue culture system disclosed herein, wherein primary sets of lymphocytes are plated at about a concentration of from about 0.001 to about 10 million cells per milliliter into one or more compartments of the cell culture unit and then allowed to grow to a confluent layer on surface area of from about 1 to about 200 squared centimeters. In some embodiments, the method further comprises removing one or more partitions to allow the cells to grow in a second compartment until confluence, when again, optionally, another partition may successively be removed to allow for more surface are for expanded culture. In some embodiments the method of culturing further comprises repeating the step of removing a partition for each of the compartments into which cells should grow. In some embodiments, the cell culture unit comprises 3, 4, 5, 6, 7, 8, 9, 10, 11, 12 or more partitions each of which corresponding to the physical barrier between a second and third compartment, between a third and fourth compartment, between a fourth and fifth compartment, between a fifth and sixth compartment, between a sixth and seventh compartment, between a seventh and eighth compartment, between an eighth and ninth compartment, between a ninth and tenth compartment, between a tenth and eleventh compartment, and/or between an eleventh and twelfth compartment, respectively.

In some embodiments, one or more of the partitions comprise an interior portion, a frame portion and an exterior portion. The interior portion of the partition is positioned in the closed portion of the system; the frame portion spans a wall of the culture system separating the interior of the culture system to the exterior of the system; and the exterior portion is positioned outside of the system. In some embodiments, a seal operably fits around the frame portion of one or more of the partitions such that removal of the partition does not introduce pathogens to and/or does not expose the environment outside of the tissue culture system to the interior of the tissue culture system.

In some embodiments, the cell density of each compartment is from about 0.1 to about 10 million cells per mL of cell culture media. In some embodiments, the cell density of each compartment is from about 0.1 to about 10 million cells per mL of cell culture media. In some embodiments, the cell density of each compartment is from about 0.5 to about 10 million cells per mL of cell culture media. In some embodiments, the cell density of each compartment is from about 1.0 to about 10 million cells per mL of cell culture media. In some embodiments, the cell density of each compartment is from about 2 to about 10 million cells per mL of cell culture media. In some embodiments, the cell density of each compartment is from about 3 to about 10 million cells per mL of cell culture media. In some embodiments, the cell density of each compartment is from about 4 to about 10 million cells per mL of cell culture media. In some embodiments, the cell density of each compartment is from about 5 to about 10 million cells per mL of cell culture media. In some embodiments, the cell density of each compartment is from about 6 to about 10 million cells per mL of cell culture media. In some embodiments, the cell density of each compartment is from about 7 to about 10 million cells per mL of cell culture media. In some embodiments, the cell density of each compartment is from about 8 to about 10 million cells per mL of cell culture media. In some embodiments, the cell density of each compartment is from about 9 to about 10 million cells per mL of cell culture media. In some embodiments, the cell density of each compartment is from about 0.1 to about 20 million cells per mL of cell culture media. In some embodiments, the cell density of each compartment is from about 0.1 to about 50 million cells per mL of cell culture media.

In some embodiments, the systems disclosed herein comprise a cell density of from about 0.01 million to about 10 million cells per square centimeter. In some embodiments, the systems disclosed herein comprise a cell density of from about 0.03 million to about 5 million cells per square centimeter. In some embodiments, the systems disclosed herein comprise a cell density of from about 0.07 million to about 5 million cells per square centimeter. In some embodiments, the systems disclosed herein comprise a cell density of from about 0.03 million to about 5 million cells per square centimeter. In some embodiments, the systems disclosed herein comprise a cell density of from about 0.001 million to about 5 million cells per square centimeter. In some embodiments, the systems disclosed herein comprise a cell density of from about 0.002 million to about 4 million cells per square centimeter. In some embodiments, the systems disclosed herein comprise a cell density of from about 0.003 million to about 5 million cells per square centimeter of surface area of cell reactor surface. In some embodiments, the systems disclosed herein comprise a cell density of from about 0.004 million to about 5 million cells per square centimeter of surface area of cell reactor surface. In some embodiments, the systems disclosed herein comprise a cell density of from about 0.005 million to about 5 million cells per square centimeter of surface area of cell reactor surface. In some embodiments, the systems disclosed herein comprise a cell density of from about 0.006 million to about 5 million cells per square centimeter of surface area of cell reactor surface. In some embodiments, the systems disclosed herein comprise a cell density of from about 0.007 million to about 5 million cells per square centimeter of surface area of cell reactor surface. In some embodiments, the systems disclosed herein comprise a cell density of from about 0.001 million to about 4 million cells per square centimeter of surface area of cell reactor surface. In some embodiments, the systems disclosed herein comprise a cell density of from about 0.001 million to about 3 million cells per square centimeter of surface area of cell reactor surface. In some embodiments, the systems disclosed herein comprise a cell density of from about 0.003 million to about 3 million cells per square centimeter of surface area of cell reactor surface.

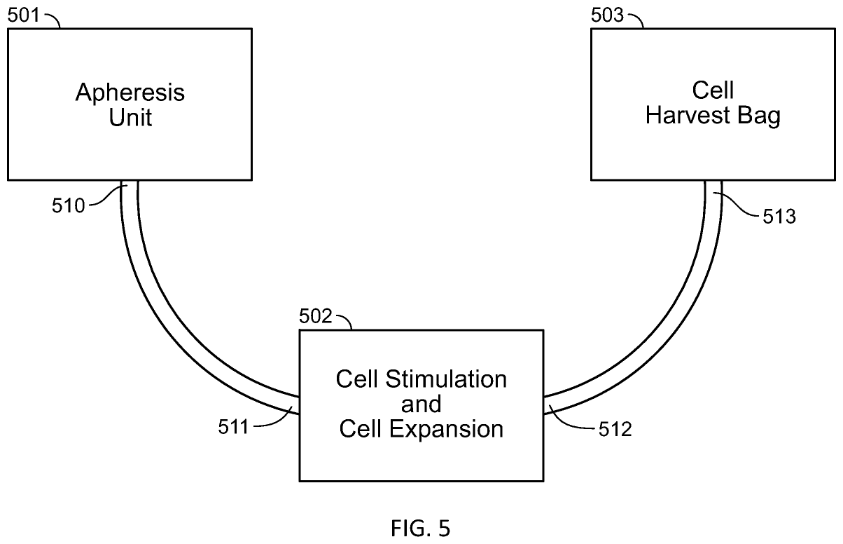

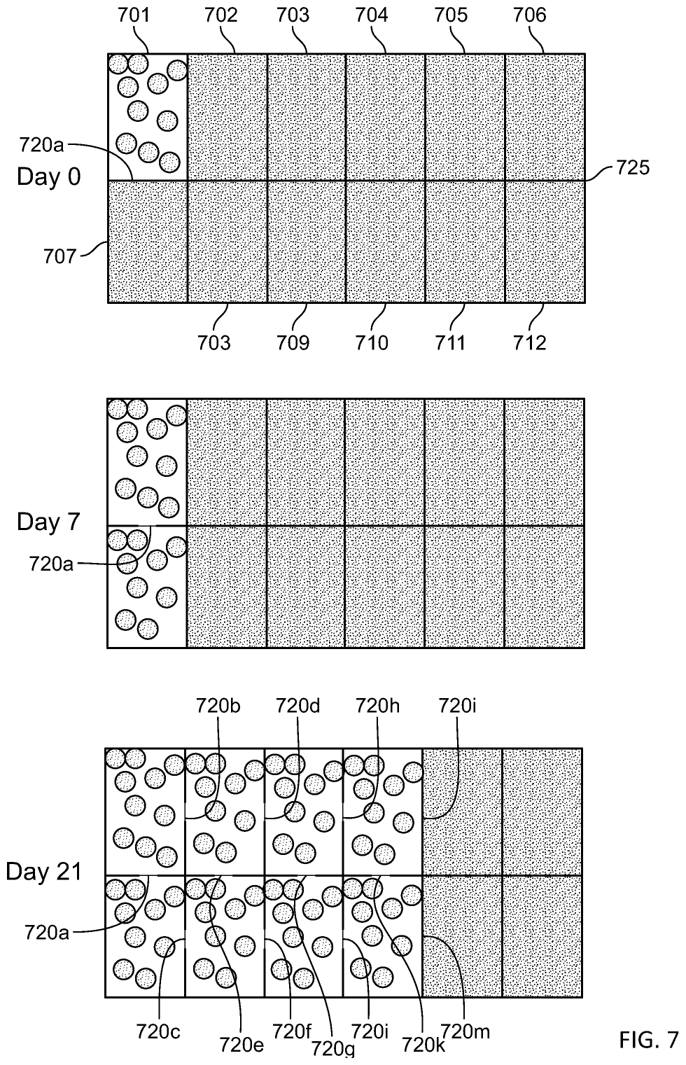

FIG. 7 depicts a two dimensional model of cell culture unit which can be operably attached to an apheresis unit such as depicted in FIG. 5. The perspective of the drawing is depicted as a view from above and shows three panels on the top, middle and bottom of the page. Each panel shows the same cell culture unit comprising multiple (twelve) compartments (701, 702, 703, 704, 705, 706, 707, 708, 709, 710, 711, and 712). The volume of the twelve compartments are defined by partitions (depicted in the middle panel as 720a-720m) that run along a lateral y axis and a longitudinal x axis (defined by partition structure 725 that is subdivided into removable section). The multi-chamber tissue culture unit of FIG. 7 allows for expansion of stimulated T cell populations after stimulation a time period sufficient for proliferation or expansion of the cells to a certain cell density has elapsed. In the top panel, cells that are stimulated in a first compartment are allowed to grow for about 7 days,m after which partition (720a) is removed. Under conditions sufficient for growth (such as exposure to CO₂ at 5% and temperatures between about 35 and 39 degrees Celsius) and cell culture media disclosed herein, the T cell populations are capable of expanding into a second compartment (707). Cells may be harvested at this point or left to growth further with removal of any one or plurality of other removable partitions (720c, 720d, 720e, 720f, 720g, 720h, 720i, 720j, 720k) are removed to allow for growth into a third, fourth, fifth, sixth, seventh and eighth compartment (703, 704, 705, 706, 707 and 708, respectively). At twenty-on days, the FIG. 7 depicts the first eight compartments exposed to culture while compartment 709, 710, 711, and 712 are left closed by partitions 720l and 720m. In some embodiments, continuous media perfusion over the surface is accomplished by a cell media pump and reservoir (not depicted). Adherent cells can be removed at Day 21 (lower panel) by exposure of the cells to enzymes that remove adhesion points of the cells the plastic. After exposure cells can be harvested by draining the non-adherent T cell populations from the cell culture unit into the harvest bag in fluid communication to the unit (not depicted in FIG. 7, but depicted in an embodiment in FIG. 5).

### Characterizing the T-Cell Subpopulation

T-cell subpopulations of some embodiments are likely to be made up of different lymphocytic cell subsets, for example, a combination of CD4+ T-cells, CD8+ T-cells, CD3+/CD56+ Natural Killer T-cells (CD3+ NKT), and TCR γδ T-cells (γδ T-cells). In particular, the T-cell subpopulation likely include at least CD4+ T-cells and CD8+ T-cells that have been primed and are capable of targeting a single specific TAA for tumor killing and/or cross presentation. The T-cell subpopulation may further comprise activated γδ T-cells and/or activated CD3+/CD56+ NKT cells capable of mediating anti-tumor responses. Accordingly, the T-cell subpopulation may be further characterized by determining the population of various lymphocytic subtypes, and the further classification of such subtypes, for example, by determining the presence or absence of certain clusters of differentiation (CD) markers, or other cell surface markers, expressed by the cells and determinative of cell subtype.

In one embodiment, the T-cell subpopulation may be analyzed to determine CD8+ T-cell population, CD4+, T-cell population, γδ T-cell population, NKT-cell population, and other populations of lymphocytic subtypes. For example, the population of CD4+ T-cells within the T-cell subpopulation may be determined, and the CD4+ T-cell subtypes further determined. For example, the CD4+ T-cell population may be determined, and then further defined, for example, by identifying the population of T-helper 1 (Th1), T-helper 2 (Th2), T-helper 17 (Th17), regulatory T cell (Treg), follicular helper T-cell (Tfh), and T-helper 9 (Th9). Likewise, the other lymphocytic subtypes comprising the T-cell subpopulation can be determined and further characterized.

In addition, the T-cell subpopulation can be further characterized, for example, for the presence, or lack thereof, of one or more markers associated with, for example, maturation or exhaustion. T cell exhaustion (Tex) is a state of dysfunction that results from persistent antigen and inflammation, both of which commonly occur in tumor tissue. The reversal or prevention of exhaustion is a major area of research for tumor immunotherapy. Tex cell populations can be analyzed using multiple phenotypic parameters, either alone or in combination. Hallmarks commonly used to monitor T cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

The T-cell subpopulations of the described compositions described herein can be subjected to further selection, if desired. For example, a particular T-cell subpopulation for inclusion in a TVM composition described herein can undergo further selection through depletion or enriching for a subpopulation. For example, following priming, expansion, and selection, the cells can be further selected for other cluster of differentiation (CD) markers, either positively or negatively. For example, following selection of for example CD4+ T-cells, the CD4+ T-cells can be further subjected to selection for, for example, a central memory T-cells (Tcm). For example, the enrichment for CD4+ Tcm cells comprises negative selection for cells expression a surface marker present on naïve T cells, such as CD45RA, or positive selection for cells expressing a surface marker present on Tcm cells and not present on naïve T-cells, for example CD45RO, CD62L, CCR7, CD27, CD127, and/or CD44. In addition, the T-cell subpopulations described herein can be further selected to eliminate cells expressing certain exhaustion markers, for example, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains)

Methods for characterizing lymphocytic cell subtypes are well known in the art, for example flow cytometry, which is described in Pockley et al., Curr Protoc Toxicol. 2015 Nov. 2; 66:18.8.1-34, which is incorporated herein by reference.

The time period sufficient for the T-cells to proliferate varies depending on, e.g., the features of the system and the type of T-cell population. For example, CD45A+ T-cells are typically allowed to grow for a time period sufficient to proliferate into a total cell number of from about 1×10⁹ to about 1×10¹² cells, including all values therein, such as for example about 1×10¹⁰ cells and about 1×10¹¹ cells.

In some embodiments, the T-cells can be co-cultured with one or a plurality of dendritic cells. In such embodiments, the one or plurality of dendritic cells can present on their surface at least one antigen to contact one or plurality of T-cells for a period of time sufficient to stimulate a T-cell response against the at least one antigen. Dendritic cells, if present, can be from the same subject as the T-cells or can be from a different subject or source.

The T-cells cultured using the present system can be harvested through any method, e.g., in a closed system as described elsewhere herein.

Tumor Associated Antigens (TAAs)

Tumor Associated Antigens (TAAs) are antigens that are highly correlated with certain tumor cells. In some embodiments, TAAs can be classified into tissue differentiation antigens (e.g. MART-1, gp100, CEA, CD19); tumor germline (“tumor-testis”) antigens (e.g. NY-ESO1, MAGE-A3); normal proteins overexpressed by cancer cells (e.g. hTERT, EGFR, mesothelin); viral proteins (e.g. HPV, EBV, MCC) and tumor-specific mutated antigens (e.g. Mum-1, B-catenin, CDK4, ERBB2IP). The disclosure relates to compositions and methods herein comprising CD45A+ T cells stimulated by one or a plurality of TAAs.

In certain embodiments, the TAA is expressed in a cancer selected from acute lymphoblastic leukemia (ALL), ACUTE myeloid leukemia (AML), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumors (GTT), hairy cell leukaemia, head and neck cancer, hodgkin lymphoma, kidney cancer, laryngeal cancer, leukaemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

Antigens used for immunotherapy should be selected based on either uniqueness to tumor cells, greater expression in tumor cells as compared to normal cells, or ability of normal cells with antigen expression to be adversely affected without significant compromise to normal cells or tissue. As a non-limiting example, Wilms tumor gene (WT1) is found in post-natal kidney, pancreas, fat, gonads and hematopoietic stem cells. In healthy hematopoietic stem cells WT1 encodes a transcription factor, which regulates cell proliferation, cell death and differentiation. WT1 is overexpressed in Wilms tumor, soft tissue sarcomas, rhabdomyosarcoma, ovarian, and prostate cancers. The WT1 gene was initially identified as a tumor suppressor gene due to its inactivation in Wilms' tumor (nephroblastoma), the most common pediatric kidney tumor. However, recent findings have shown that WT1 acts as an oncogene in ovarian and other tumors. In addition, several studies have reported that high expression of WT1 correlates with the aggressiveness of cancers and a poor outcome in leukemia, breast cancer, germ-cell tumor, prostate cancer, soft tissue sarcomas, rhabdomyosarcoma and head and neck squamous cell carcinoma. There are several studies describing WT1 expression in ovarian cancers. A positive expression has been primarily observed in serous adenocarcinoma, and WT1 is more frequently expressed in high-grade serous carcinoma, which stands-out from other sub-types due to its aggressive nature and because it harbors unique genetic alterations. Patients with WT1-positive tumors tend to have a higher grade and stage of tumor.

Preferentially expressed antigen of melanoma (PRAME), initially identified in melanoma, has been associated with other tumors including neuroblastoma, osteosarcoma, soft tissue sarcomas, head and neck, lung and renal cancer including Wilms tumor. In neuroblastoma and osteosarcoma, PRAME expression was associated with advanced disease and a poor prognosis. PRAME is also highly expressed in leukemic cells and its expression levels are correlated with relapse and remission. The function in healthy tissue is not well understood, although studies suggest PRAME is involved in proliferation and survival in leukemia cells.

Survivin is highly expressed during normal fetal development but is absent in most mature tissues. It is thought to regulate apoptosis and proliferation of hematopoietic stem cells. Overexpression of survivin has been reported in almost all human malignancies including bladder cancer, lung cancer, breast cancer, stomach, esophagus, liver, ovarian cancers and hematological cancers. Survivin has been associated with chemotherapy resistance, increased tumor recurrence and decreased survival.

Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies). Although they are preferentially expressed by tumor cells, TAAs are sometimes found in normal tissues. However, their expression differs from that of normal tissues by their degree of expression in the tumor, alterations in their protein structure in comparison with their normal counterparts or by their aberrant subcellular localization within malignant or tumor cells.

Examples of oncofetal tumor associated antigens include Carcinoembryonic antigen (CEA) (GenBank Accession No. GenBank: M17303.1), immature laminin receptor, and tumor-associated glycoprotein (TAG) 72. Examples of overexpressed/accumulated include BING-4, calcium-activated chloride channel (CLCA) 2, Cyclin B1, 9D7, epithelial cell adhesion molecule (Ep-Cam) (NCBI Reference Sequence: NM_002354.2), EphA3, Her2/neu, telomerase, mesothelin (NCBI Reference Sequence: NM_013404.4), orphan tyrosine kinase receptor (ROR1), stomach cancer-associated protein tyrosine phosphatase 1 (SAP-1), and survivin.

Examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10 (GenBank: AF116194.1), NY-ESO-1 (NCBI Reference Sequence: NM_001327.2), L antigen (LAGE) 1, Melanoma antigen preferentially expressed in tumors (PRAME), and synovial sarcoma X (SSX) 2. Examples of lineage restricted tumor antigens include melanoma antigen recognized by T cells-1/2 (Melan-A/MART-1/2), Gp100/pme117 (NCBI Reference Sequence: NM_001320122.1), tyrosine-related protein (TRP) 1 and 2, P. polypeptide, melanocortin 1 receptor (MC1R), and prostate-specific antigen. Examples of mutated tumor antigens include β-catenin, breast cancer antigen (BRCA) 1/2, cyclin-dependent kinase (CDK) 4, chronic myelogenous leukemia antigen (CML) 66, fibronectin, p53, Ras, and TGF-βRII. An example of a post-translationally altered tumor antigen is mucin (MUC) 1. Examples of idiotypic tumor antigens include immunoglobulin (Ig) and T cell receptor (TCR).

In some embodiments, the antigen associated with the disease or disorder is selected from the group consisting of CD19 (GenBank: AH001873.2), CD20 (GenBank: AH003353.2), CD22, hepatitis B surface antigen, anti-folate receptor, CD23, CD24, CD30 (GenBank: AH008756.3), CD33, CD38, CD44, EGFR, EGP-2, EGP-4, OEPHa2, ErbB2, 3, or 4, FBP, fetal acetylcholine receptor, HMW-MAA, IL-22R-alpha, IL-13R-alpha, kdr, kappa light chain, Lewis Y, MUC16 (CA-125), PSCA, NKG2D Ligands, oncofetal antigen, VEGF-R2, PSMA, estrogen receptor, progesterone receptor, ephrinB2 (NCBI Reference Sequence: NM_004093.3), CD123 (NCBI Reference Sequence: NM_002183.3), CS-1, c-Met and/or biotinylated molecules, and/or molecules expressed by HIV, HCV, HBV or other pathogens.

Exemplary tumor antigens include at least the following: carcinoembryonic antigen (CEA(GenBank Accession No. GenBank: M17303.1)) for bowel cancers; CA-125 for ovarian cancer; MUC1 (GenBank: X80761.1) or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CA19-9 for colorectal, bile duct, and pancreatic cancer; chromogranin A for lung and prostate cancer; TA90 for melanoma, soft tissue sarcomas, and breast, colon, and lung cancer.

Examples of TAAs are known in the art, for example in N. Vigneron, “Human Tumor Antigens and Cancer Immunotherapy,” BioMed Research International, vol. 2015, Article ID 948501, 17 pages, 2015. doi:10.1155/2015/948501; Ilyas et al., J Immunol. (2015) Dec. 1; 195(11): 5117-5122; Coulie et al., Nature Reviews Cancer (2014) volume 14, pages 135-146; Cheever et al., Clin Cancer Res. (2009) Sep. 1; 15(17):5323-37, which are incorporated by reference herein in its entirety.

Examples of oncoviral TAAs include human papilloma virus (HPV) L1, E6 and E7, Epstein-Barr Virus (EBV) Epstein-Barr nuclear antigen (EBNA), EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis B x antigen (HBxAg), hepatitis C core antigen (HCV core Ag), Human T-Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV-1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GM1b, GD1c, GM3, GM2, GM1a, GD1a, GT1a, GD3, GD2, GD1b, GT1b, GQ1b, GT3, GT2, GT1c, GQ1c, and GP1c. Examples of ganglioside derivatives include, for example, 9-O—Ac-GD3, 9-O—Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2.

Recent analyses of The Cancer Genome Atlas (TCGA) datasets have linked the genomic landscape of tumors with tumor immunity, implicating neoantigen load in driving T cell responses (Brown et al., Genome Res. 2014 May; 24(5):743-50, 2014) and identifying somatic mutations associated with immune infiltrates (Rutledge et al., Clin Cancer Res. 2013 Sep. 15; 19(18):4951-60, 2013). Rooney et al. (2015 Jan. 15; 160(1-2):48-61) suggest that neoantigens and viruses are likely to drive cytolytic activity, and reveal known and novel mutations that enable tumors to resist immune attack. Thus, in certain embodiments, the cancer is a cancer associated with an oncogenic virus, for example Epstein Barr virus (EBV), hepatitis B and C (HBV and HCV), human papilloma virus (HPV), Kaposi sarcoma virus (KSV), and polyoma viruses. In other certain embodiments, the cancer is a cancer where retrovirus epitopes are identified. Cancers which are associated with a virus and which may be treated using the methods of the invention include, but are not limited to, cervical cancer, head and neck cancer, lymphomas, and kidney clear cell carcinoma.

### Viral Associated Antigens (VAAs)

A viral antigen is a toxin or other substance given off by a virus which causes an immune response in its host. Viral antigens are protein in nature, strain-specific, and closely associated with the virus particle. A viral antigen is a protein encoded by the viral genome. A viral protein is an antigen specified by the viral genome that can be detected by a specific immunological response. The disclosure relates to compositions and methods herein comprising CD45A+ T cells stimulated by one or a plurality of VAAs.

Each virus has its own viral-associated antigens. Examples of antigens to cytomegalovirus (CMV) include immediate-early protein 1 (IE-1), immediate-early protein 2 (IE-2), 65 kDa phosphoprotein (pp65). Examples of antigens to Epstein-Barr Virus (EBV) include the Epstein-Barr Nuclear Antigen (EBNA) family, which includes EBNA-leader protein (EBNA-LP), EBNA1, EBNA2, EBNA3a, EBNA3b, EBNA3c; latent membrane protein (LMP) family, which includes LMP1 and LMP2; envelope glycoprotein GP350/GP340; secreted protein BARF1; mRNA export factor EB2 (BMLF1); DNA polymerase processivity factor (BMRF1) and trans-activator protein (BZLF1). Examples of antigens to human adenovirus (HAdV) include the hexon protein of Human adenovirus 3 (HAdV-3) and the penton protein of Human adenovirus 5 (HAdV-5). Examples of antigens to BK polyomavirus include capsid protein VP-1, capsid protein VP-2, large T antigen, and small T antigen. Examples of antigens to Human herpesvirus 6 (HHV-6) include proteins U14, U54 and U90. Examples of antigens to respiratory syncytial virus (RSV) include the fusion glycoprotein (F), major surface glycoprotein G, small hydrophobic protein (SH), and nucleocapsid (N) protein. Examples of antigens to human influenza include matrix protein (MP) 1, matrix protein (MP) 2, nucleocapsid protein (NP) 1, neuroaminidase, hemagglutinin (HA). Examples of antigens to human papillomavirus (HPV) include protein E4, protein E5, protein E6, protein E7, late major capsid protein (L) 1, replication protein E1, and replication protein E2. Examples of antigens to human immunodeficiency virus (HIV) include envelope glycoprotein gp160 (Env), Gag polyprotein, Nef protein, and Pol polyprotein.

In one aspect, the disclosure features a method of manufacturing T cells that are specific for tumor associated antigens (TAAs) or viral associated antigens (VAAs) where the donor is seronegative (such as cord blood or adult serongegative donors), the expanded T cell product will be derived from the naïve T cell population instead of the memory T cell population, which has been the source of T cells in many other cellular therapy protocols, where the method comprises isolation of naïve T cells, expansion of naïve T cells and harvest. The disclosed methods advantageously provide a large scale method of manufacturing T cells that are specific for TAAs or VAAs. A large scale method of manufacturing includes volumes of about 100 mL or more, with the number of cells 10 exceed about 500,000, about 1,000,000, about 10,000,000, about 100,000,000, about 1,000,000,000, about 10,000,000,000, about 100,000,000,000.

### CD45RA Selection

In some embodiments, CD45RA can be selected for by isolating fresh or frozen PBMC and setting aside at least about 5×10⁵ PBMCs for pre-selection flow cytometry to determine % CD3/CD45RA+ lymphocytes. PBMCs can then be centrifuged and resuspended in, e.g., a ClinMAX® incubation buffer. After incubation, PBMCs can then again be centrifuged and resuspended in solution comprising anti-CD45RA antibodies (e.g., comprising anti-CD45RA microbeads). After another incubation and blocking and washing steps as needed, the CD45RA+PMBCs can be isolated by flow cytometry using any suitable flow cytometry device as instructed, e.g., with CliniMACS® system. At least about 1×10⁵ PBMCs should be set aside for post-selection flow cytometry.

### Apheresis

One type of extracorporeal blood processing is an apheresis procedure in which blood is removed from a donor or patient, directed to a blood component separation device (e.g., centrifuge), and separated into various blood component types (e.g., red blood cells, white blood cells, platelets, plasma) for collection or therapeutic purposes. One or more of these blood component types are collected (e.g., for therapeutic purposes), while the remainder are returned to the donor or patient.

In one embodiment the blood is processed as described herein prior to transportation.

In one embodiment the blood sample or processed blood sample is transported at ambient temperature, for example above 4° C. and below about 30° C.

In one embodiment the blood sample or processed blood sample is filled into a container, such as bag, comprising two chambers, wherein one chamber contains additives, such as preservatives and/or anticoagulants and the blood or processed blood is filled into the second chamber, after which a seal between the first and second chamber is broken and the contents of the two chambers are mixed. Culturing cells as employed herein is intended to refer to activating and expanding and/or differentiating cells in vitro.

In one embodiment, the monocyte and leukocyte fractions are obtained from the blood or apheresis product by Ficoll density gradient separation known to those skilled in the art. Ficoll density gradient separation employs a synthetic sucrose polymer the concentration of which varies through the solution to exploit the separation of different cells during sedimentation. Suitable reagents are available, for example from GE Healthcare, such as Ficoll PAQUEPLUS.

In another embodiment, an apheresis system is used. An apheresis system generally includes a blood component separation device (e.g., a membrane-based separation device, a rotatable centrifuge element, such as a rotor, which provides the forces required to separate blood into its various blood component types (e.g., red blood cells, white blood cells, platelets, and plasma)). In one embodiment, the separation device includes a channel which receives a blood processing vessel. Typically, a healthy human donor or a patient suffering from some type of illness (donor/patient) is fluidly interconnected with the blood processing vessel by an extracorporeal tubing circuit, and preferably the blood processing vessel and extracorporeal tubing circuit collectively define a closed, sterile system. When the fluid interconnection is established, blood may be extracted from the donor/patient and directed to the blood component separation device such that at least one type of blood component may be separated and removed from the blood, either for collection or for therapy.

In one embodiment, a blood apheresis system allows for a continuous blood component separation process. Generally, whole blood is withdrawn from a donor/patient and is provided to a blood component separation device where the blood is separated into the various component types and at least one of these blood component types is removed from the device. These blood components may then be provided for subsequent use by another or may undergo a therapeutic treatment and be returned to the donor/patient.

In one exemplary blood apheresis system, blood is withdrawn from the donor/patient and directed through a disposable set which includes an extracorporeal tubing circuit and a blood processing vessel and which defines a completely closed and sterile system. The disposable set is mounted on the blood component separation device which includes a pump/valve/sensor assembly for interfacing with the extracorporeal tubing circuit, and a channel assembly for interfacing with the disposable blood processing vessel.

The channel assembly includes a channel housing which is rotatably interconnected with a rotatable centrifuge rotor assembly which provides the centrifugal forces required to separate blood into its various blood component types by centrifugation. The blood processing vessel is interfitted with the channel housing. Blood thus flows from the donor/patient, through the extracorporeal tubing circuit, and into the rotating blood processing vessel. The blood within the blood processing vessel is separated into various blood component types and at least one of these blood component types (e.g., platelets, plasma, red blood cells) is continually removed from the blood processing vessel. Blood components which are not being retained for collection or for therapeutic treatment (e.g., red blood cells, white blood cells, plasma) are also removed from the blood processing vessel and returned to the donor/patient via the extracorporeal tubing circuit. In one embodiment, blood apheresis systems are described in WO1996040322A3, U.S. Pat. No. 7,497,944B2, U.S. Pat. No. 5,653,887A, incorporated by reference in their entireties herein.

In some embodiments, a system for processing blood components may comprise a separation chamber including a chamber interior in which blood components are centrifugally separated and an outlet port for passing at least some centrifugally separated blood components from the chamber interior. A flow path may be in flow communication with the outlet port of the separation chamber. The apparatus may further comprise a filter including a filter inlet in flow communication with the flow path, a porous filtration medium configured to filter at least some of at least one blood component (e.g., leukocytes, platelets, and/or red blood cells) from centrifugally separated blood components passed to the filter via the flow path, and a filter outlet for filtered blood components. The system may further comprise a rotor configured to be rotated about an axis of rotation. The rotor may comprise a first portion configured to receive the separation chamber and a second portion configured to receive the filter, wherein the first and second portions may be positioned with respect to one another so that when the separation chamber is received in the first portion and the filter is received in the second portion, the filter is closer than the interior of the separation chamber to the axis of rotation. The system may be configured so that the rotor rotates during filtering of at least one blood component via the filter.

In some embodiments, the starting product will be an apheresis mononuclear cell product that will be collected from a non-mobilized donor. In some embodiments, the starting product will be a peripheral blood mononuclear cell (PBMC). PSMCs consist of lymphocytes (T cells, B cells, NK cells) and monocytes. In humans, lymphocytes make up the majority of the PBMC population, followed by monocytes, and only a small percentage of dendritic cells.

The product will be collected from a health donor or patient and apheresed using a collection machine

In one embodiment, the Spectra Optia (TerumoBCT) system is used for apheresis. This system allows efficient peripheral blood stem cell collections. With this procedure, mononuclear cells (MNCs) are collected, including monocytes, lymphocytes, CD34+ and dendritic cells.

In one embodiment, the Elutra system (TeromoBCT) is used to process the aphereisis product.

The Elutra system passes fluid through the cell layer established within a centrifugal field inside the separation chamber. By varying the flow of fluid in the opposite direction to the centrifugal force, the system aligns and collects particles according to size (smallest to largest) and density (lower to higher). Using both size and density as separation factors increases the resolution of cell separation compared to what is achieved with traditional centrifugation.

Compositions and methods disclosed herein comprise fraction with high cell numbers of isolated T cells. In some embodiments, cell culture step or harvest step can load or result in cell number from about 1×10⁹ to 2×10¹⁰ or more. In some embodiments, the receovery of cells can yiled from about 1 to about 5×10⁹ monocytes (in fraction after apheresis) and about 8×10⁹ (in fraction 1 or 2 after apheresis). In some embodiments the methods disclosed in Stroncek et al. Journal of Translational Medicine. 2014 are employed, the full contents of which are incorporated by reference.

### Generation of Dendritic Cells

Dendritic cells can be differentiated from monocyte fraction by culture in GM-CSF and IL-4. The dendritic cells can then be matured using GM-CSF, I L-4, I L-4β, IL-6, TN F-a and PGE-1 or PGE-2 (PGE=Prostaglandin E).

In some embodiments, to generate dendritic cells from the monocyte fraction bag, the monocyte fraction will be plated into a closed system bioreactor such as the Quantum Cell Expansion System.

Exemplary cell expansion systems (CES) are described in U.S. Pat. Nos. 8,906,688, and 9,260,698, incorporated by reference in their entireties herein.

The cell growth chamber of the cell expansion system generally includes a hollow fiber membrane including a plurality of semi-permeable hollow fibers 50 separating first and second fluid flow paths.

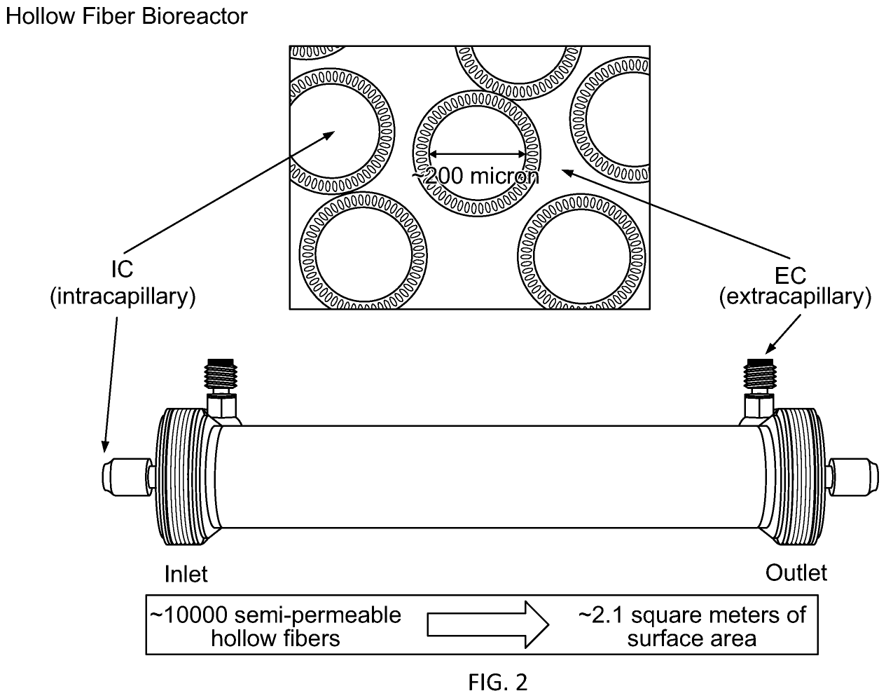

An exemplary cell growth chamber is depicted in FIG. 2 of U.S. Pat. No. 8,906,688, which depicts a cut-away side view of the hollow fiber cell growth chamber 24. Hollow fibers or membrane 50 are disposed within cell growth chamber housing 52. The housing has first and second ends which define a longitudinal axis through the housing. The housing 52 further includes four openings, or ports: inlet port 22, outlet port 28, inlet port 42, and outlet port 40.

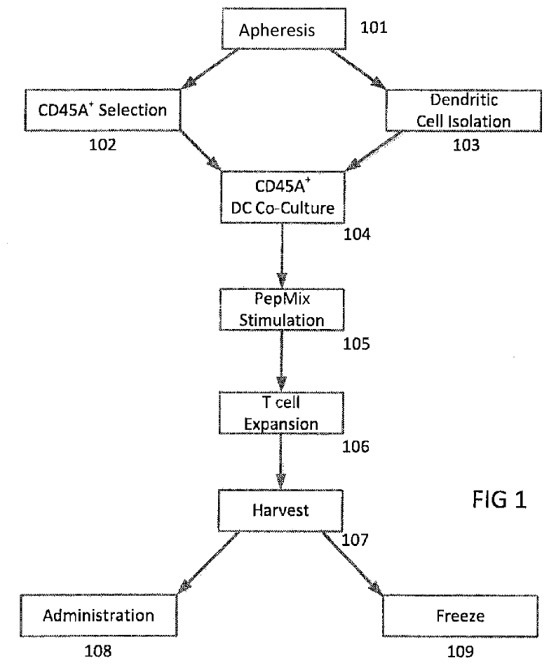

Fluid in the first fluid flow path 16 (see FIG. 1 of US80906688) enters cell growth chamber 24 through inlet port 22, passes into and through the intracapillary space of the hollow fibers and out of cell growth chamber 24 through outlet port 28. The terms “hollow fiber,” “hollow fiber capillary,” and “capillary” are used interchangeably. A plurality of hollow fibers are collectively referred to as a “membrane.” Fluid in the second fluid flow path 34 (FIG. 1) enters the cell growth chamber through inlet port 42, comes in contact with the outside of the hollow fibers, and exits cell growth chamber 24 via outlet port 40.

Cells to be expanded are contained within the first fluid flow path 16 on the IC side of the membrane. The term “fluid” may refer to gases and/or liquids. In an embodiment, a fluid containing liquid such as cell growth media is flown into the first fluid flow path 16, while a fluid containing gas such as at least oxygen is flown into the second fluid flow path 34. The gas diffuses through the membrane from the EC space into the IC space. The liquid however, must remain in the IC space and not leak through the membrane into the EC space.

Although cell growth chamber housing 52 is depicted as cylindrical in shape, it can have any other shape known in the art. Cell growth chamber housing 52 can be made of any type of biocompatible polymeric material.

Those of skill in the art will recognize that the term cell growth chamber does not imply that all cells being grown or expanded in a CES are grown in the cell growth chamber. In many embodiments, adherent cells can adhere to membranes disposed in the growth chamber, or may grow within the associated tubing. Non-adherent cells (also referred to as “suspension cells”) can also be grown.

The ends of hollow fibers 50 can be potted to the sides of the cell growth chamber by a connective material (also referred to herein as “potting” or “potting material”). The potting can be any suitable material for binding the hollow fibers 50, provided that the flow of media and cells into the hollow fibers is not obstructed and that liquid flowing into the cell growth chamber through the IC inlet port flows only into the hollow fibers. Exemplary potting materials include, but are not limited to, polyurethane or other suitable binding or adhesive components. In various embodiments, the hollow fibers and potting may be cut through perpendicular to the central axis of the hollow fibers at each end to permit fluid flow into and out of the IC side. End caps 54 and 56 are disposed at the end of the cell growth chamber.

The hollow fibers are configured to allow cells to grow in the IC space of the fibers. The fibers are large enough to allow cell adhesion in the lumen without substantially impeding the flow of media through the hollow fiber lumen.

In various embodiments, cells can be loaded into the hollow fibers by any of a variety of methods, including by syringe. The cells may also be introduced into the cell growth chamber from a fluid container, such as a bag, which may be fluidly associated with the IC side of the cell growth chamber.

Any number of hollow fibers can be used in a cell growth chamber, provided the hollow fibers can be fluidly associated with the inlet and outlet ports of the cell growth chamber.

The hollow fibers may be made of a material which will prevent the liquid contained in the IC space from leaking through the membrane into the EC space, yet must also allow the gasses contained in the EC space to diffuse through the membrane into the IC space. The outside of the fibers therefore may be hydrophobic, while the inside of the fibers may be hydrophilic.

Porous polymeric material which may be used includes polycarbonate, polyethylene sheets containing discrete holes to allow gas through, polypropylene and polytetrafluoroethylene (Teflon). Non-porous material such as silicone may also be used. The material used may be solely of one type, or may be a combination of materials, for example, one type on the inside of the hollow fibers and another type on the outside. The material must be capable of being made into hollow fibers.

In another embodiment, the hollow fibers may be coated with a substance or combinations of substances to make the surfaces hydrophobic and hydrophilic.

The material must also be capable of binding to certain types of cells, such as adherent stem cells (e.g. MSCs). Depending upon the type of cells to be expanded in the oxygenated cell growth chamber, the surface of the fibers in direct contact with the cells to be expanded may be treated with a substance such as fibronectin, platelet lysate or plasma to enhance cell growth and/or adherence of the cells to the membrane.

Dendritic cells are often referred to, by those skilled in the art, as professional antigen presenting cells. The term refers to the fact that dendritic cells are optimal in delivery the two signal activation process to T cells, i.e., in addition to presenting antigen on the cell surface, dendritic cells also provide a strong co-stimulatory signal. Both signals, stimulation by antigen presentation and co-stimulation are required to achieve T cell activation.

In some embodiments, to generate dendritic cells from the monocyte fraction bag, the monocyte fraction will be plated into a closed system bioreactor such as the Quantum Cell Expansion System. At least about 1×10⁸ to about 1×10¹⁰, at least about 1×10⁹ to about 1×10¹⁰ at least about 1×10⁸′ at least about 5×10⁸′ at least about 1×10⁹, at least about 5×10⁹′ at least about 1×10¹⁰, at least about 1×10⁹, about 2×10⁹, about 3×10⁹, about 4×10⁹, about 5×10⁹, about 6×10⁹, about 7×10⁹, about 8×10⁹, about 9×10⁹, about 10×10⁹ cells from the monocyte fraction will be added via the Cell Inlet bag on the Intracapillary (IC) line of the Quantum cell expansion system to yield a cell density of between about 1×10⁴ cells/cm² to about 1×10⁶ cells/cm², between about 1×10⁵ cells/cm² to about 1×10⁶ cells/cm², about 1×10⁴ cells/cm²′ about 5×10⁴ cells/cm², about 1×10⁵ cells/cm²′ about 5×10⁵ cells/cm², about 1×10⁶ cells/cm²′ about 5×10⁶ cells/cm², about 3.3×10⁵ cells/cm². The cells are allowed to adhere for a certain amount of time (e.g. 2-4 hours) and IL-4 and GM-CSF are added to the Quantum via the reagent bag of the IC line. Cells are re-fed after 1-2 days with the same concentration of GM-CSF and IL-4. On day 2-5, preferably on day 2, the cells will be matured using a cytokine cocktail including, but not limited to, one or more of LPS, IL-4, GM-CSF, TNF-Alpha, IL-6, and IL-1beta. 1-2 days after maturation, cells are harvested from the Quantum.

To harvest the cells, media is added at a high rate into the collection back; this will collect all non-adherent cells. To then harvest the adherent cells, the Harvest task that is pre-loaded on the device is used. The cells will be incubated with TrypLE select or a similar dissociation reagent at which point the Release Cells task will harvest all cells into the Harvest bag. If necessary, a new bag can be loaded onto the harvest line to accommodate additional volume or washes to collect all cells.

Next, the media is volume reduced, and the cells are rid of unwanted media and growth factors, and the cells are concentrated. In certain embodiments, to do this, the cells are processed on the Lovo automated cell processing system or a similar device like the Sepax; alternatively, the bag can be centrifuged and the supernatant expressed off into another bag.

In certain embodiments, the volume is reduced to about 10 to about 250 mL range, for example about 10, about 20, about 30, about 40, about 50, about 60, about 70, about 80, about 90, about 100, about 110, about 120, about 130 about 140, about 150, about 160, about 170, about 180, about 190, about 200, about 210, about 220, about 230, about 240, or about 250 mL. Next, half to three-quarters of the cells, are removed and cryopreserved to be used for a second stimulation.

To the second fraction, peptide or peptide mixture is added.

In one embodiment, peptides or peptide mixtures described herein comprise TAAs or VAAs as described herein.

In some embodiments, peptides as employed herein intended to refer to short polymers of amino acids linked by peptide bonds, wherein the peptides contain at least 2 but generally not more than 50 amino acids.

The peptides employed are sufficiently long to present one or more linear epitopes, for example are on average 7, 8, 9, 10, 11, 12, 13, 14, 15, 16, 17, 18, 19 or 20 amino acids long.

In one embodiment some of the peptides of the mixture overlap (in relation to the sequence of a single antigen), that is to say that they are from a single antigen and are arranged such that portions of the fragments and certain sequence of amino acids from the parent sequence occur in more than one peptide fragment of the mix. The overlap of the peptides means that there is redundancy in the amino acid sequence. However, this method maximises the opportunity to present epitopes from the parent antigen in an appropriate manner, particularly when epitope mapping information is not available for the parent antigen.

In one embodiment 1, 2, 3, 4, 5, 6, 7, 8, 9, 10, 11, 12, 13, 14 or 15 amino acids overlap in each peptide. In one embodiment the peptides in the libraries for each protein are 15 amino acids long and overlap by 11 amino acids so that all potential HLA class I epitopes can be presented from a protein. The peptides can be longer, for example 20 amino acids overlapping by 15 or 30 amino acids overlapping by 25.

In one embodiment the peptide mix comprises or consists of about 2 to about 1000 peptides, more specifically about 2 to about 500, for example about 2 to about 400, about 2 to about 300, about 2 to about 200 or about 2 to about 100 such as about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more peptides.

In certain embodiments, about 50 to about 200 ng of each peptide or peptide mixture is added. In some embodiments, about 50, 60, 70, 80, 90, 100, 110, 120, 130 140, 150, 160, 170, 180, 190 or about 200 ng of each peptide or peptide mixture is added. In other embodiments, 100 ng of each peptide or peptide mixture is added. In certain embodiments, about 50 to about 200 ng of each peptide or peptide mixture is added per 10 million dendritic cells. In some embodiments, about 50, 60, 70, 80, 90, 100, 110, 120, 130 140, 150, 160, 170, 180, 190 or about 200 ng of each peptide or peptide mixture is added per 10 million dendritic cells. In other embodiments, about 100 ng of each peptide or peptide mixture is added per 10 million dendritic cells. The peptides or peptide mixtures can be added using a luer lock or port on the bag. The bag will be mixed periodically and incubated with the peptides for about 30 minutes to about 2 hours, for example about 30, 40, 50, 60, 70, 80, 90, 100, 110 or 120 minutes. Once the incubation period is complete, the cells are re-loaded into the Quantum Cell Expansion System. In some embodiments, the cells are loaded with the dendritic cells at a ther1:5-1:50 ratio of dendritic cells to lymphocytes, for example a 1:5 to 1:10 ratio, a 1:5 to 1:20 ratio, a 1:5 to 1:30 ratio, a 1:5 to 1:40 ratio. In one embodiment, the cells are loaded with the dendritic cells at a 1:5 ratio of dendritic cells to lymphocytes. In one embodiment, the cells are loaded with the dendritic cells at a 1:10 ratio of dendritic cells to lymphocytes. In one embodiment, the cells are loaded with the dendritic cells at a 1:15 ratio of dendritic cells to lymphocytes. In one embodiment, the cells are loaded with the dendritic cells at a 1:20 ratio of dendritic cells to lymphocytes. In one embodiment, the cells are loaded with the dendritic cells at a 1:25 ratio of dendritic cells to lymphocytes. In one embodiment, the cells are loaded with the dendritic cells at a 1:30 ratio of dendritic cells to lymphocytes. In one embodiment, the cells are loaded with the dendritic cells at a 1:35 ratio of dendritic cells to lymphocytes. In one embodiment, the cells are loaded with the dendritic cells at a 1:40 ratio of dendritic cells to lymphocytes. In one embodiment, the cells are loaded with the dendritic cells at a 1:45 ratio of dendritic cells to lymphocytes. In one embodiment, the cells are loaded with the dendritic cells at a 1:50 ratio of dendritic cells to lymphocytes.

Cell Expansion Systems

A cell growth chamber such as the one depicted in FIG. 2 of U.S. Pat. No. 8,906,688 is operably associated with other components of a cell expansion system.

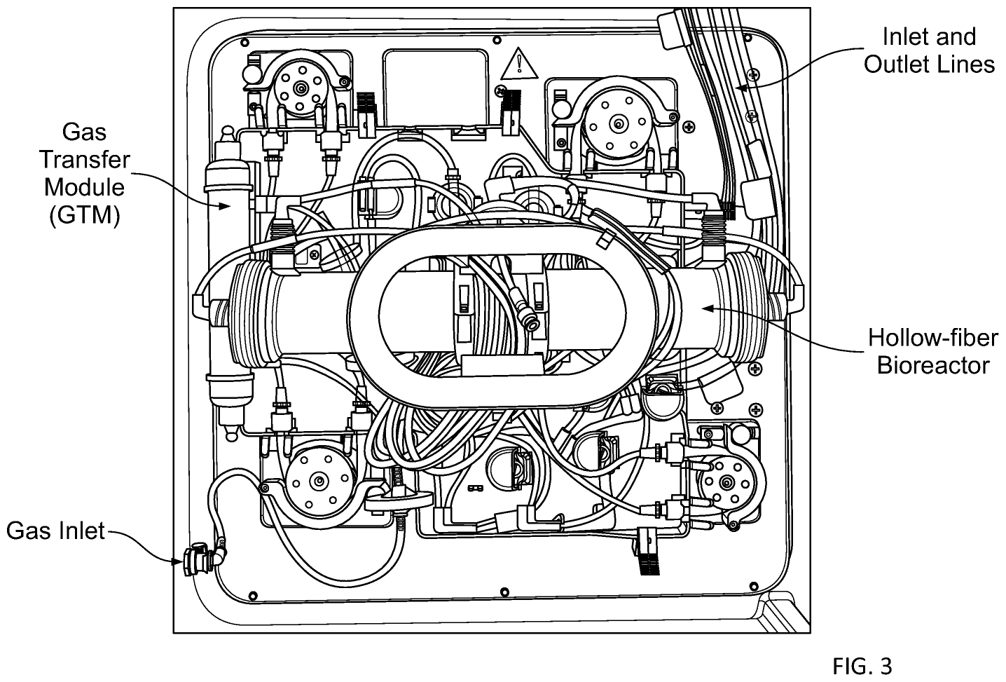

FIG. 3 of U.S. Pat. No. 8,906,688 depicts a more detailed cell expansion system 10. CES 10 includes first fluid flow path 12 and second fluid flow path 14. Fluid flow paths are constructed of tubing and tubing conduits (Tygothane, St. Globain) and operate in conjunction with valves, pumps and other components (not shown).

Outlet port 28 of cell growth chamber 24 is fluidly associated via tubing with inlet port 22, which together with cell growth chamber 24 form first fluid flow path 12. First fluid flow path 12 is configured to circulate fluid through the IC space of the cell growth chamber 24. First fluid flow path 12 is configured for fluid such as cell growth media to flow through cell growth chamber 24, pump 30, and back through cell growth chamber 24. Cells can be flushed out of cell growth chamber 24 through outlet port 28 to cell harvest bag 140 or can be redistributed back into the IC space via port 22.

CES 10 also includes second fluid flow path 14. Second fluid flow path 14 is configured to flow fluid such as gas through the EC space of the cell growth chamber. The second fluid flow path 14 connects to cell growth chamber 24 by inlet port 42, and departs cell growth chamber 24 via outlet port 40. In the embodiment shown in FIG. 3, gas flows out of gas container or tank 130 into the EC space through port 42, around the hollow fibers of CES 24 and out of the cell growth chamber via port 40. Gas which does not diffuse through the fibers into the IC space, and any carbon dioxide or other gasses which have diffused into the EC space from the IC space flows out of the cell growth chamber through outlet port 40. Gas flows through second fluid flow path at substantially atmospheric pressure. No pump or other means to actively move the gas through the second fluid flow path is needed, as the gas flowing out of tank 130 is under pressure, and once released from the tank, will passively flow at substantially atmospheric pressure. As gas is customarily stored at high pressure, a pressure regulator or orifice or nozzle (not shown) may be placed at the opening of tank 130 to help reduce the initial pressure of the gas flowing out of the tank. The pressure of the gas flowing through the membrane must be at a low enough pressure to avoid formation of gas bubbles within the cell culture chamber 24, but at a high enough pressure to avoid a drop in pressure between inlet port 42 and outlet port 40.

The concentration of gases in the cell growth chamber can be at any concentration desired. Gases diffuse across the fibers in the cell growth chamber. Filters 150 and 152 prevent contamination of the cell growth chamber with airborne contaminants as the gas flows through second fluid flow path 14.

In another embodiment (not shown), a pump could be added to the second fluid flow path 14 to pump the gas containing oxygen through the second fluid flow path. The pump could be located anywhere on second fluid flow path. Another orifice or pressure regulator could also be placed at the end 150 of second fluid flow path to control any drop in pressure which may occur along the bioreactor and to increase the pressure within the bioreactor.

Liquid media contained in first fluid flow path 12 is in equilibrium with the gases flowing across the membrane from second fluid flow path 14. The amount of gas containing oxygen entering the media can be controlled by controlling the concentration of oxygen. The mole percent (also referred to herein as “Molar concentration”) of oxygen in the gas phase before diffusing into the media is typically greater than or equal to 0%, 5%, 10% or 15%. Alternatively, the molar concentration of oxygen in the gas is equal to or less than 20%, 15%, 10% or 5%. In certain embodiments, the molar concentration of oxygen is 5%.

CES 10 includes first fluid inlet path 44. First fluid inlet path 44 includes drip chamber 80 and pump 48. Fluid media and/or cells flow from IC media container 108 and/or cell input bag 112. Each of IC fluid media container 108, vent bag 110, or cell input bag 112 are fluid media containers as discussed herein. IC media refers to media that circulates in first fluid flow path 12.

Drip chamber 80 helps prevent pockets of gas (e.g. air bubbles) from reaching cell growth chamber 24. Ultrasonic sensors (not shown) can be disposed near entrance port and exit port of drip chamber 80. A sensor at entrance port prevents fluids in drip chamber 80 from back-flowing into IC media container 108, vent bag 110, cell input bag 112, or related tubing. A sensor at the exit port stops pump 48 if gas reaches the bottom of the sensor to prevent gas bubbles from reaching the IC side of cell growth chamber 24.

Those of skill in the art will recognize that fluid in first fluid flow path 12 can flow through cell growth chamber 24 in either the same direction as fluid in second fluid flow path 14 (co-current) or in the opposite direction of second fluid flow path 14 (i.e. counter-current).

Cells can be harvested via cell harvest path 46. Cell harvest path 46 is fluidly associated with cell harvest bag 140 and first fluid circulation path 12 at junction 188. Cells from cell growth chamber 24 can be pumped via pump 30 through cell harvest path 46 to cell harvest bag 140.

Various components of the CES can be contained within an incubator (not shown). An incubator would maintain cells and media at a constant temperature.

Fluid outlet path 136 is associated with waste bag 148.

As used herein, the terms “media bag,” “vent bag” and “cell input bag” are arbitrary, in that their positions can be switched relative to other bags. For example, vent bag 110 can be exchanged with IC media container 108, or with cell bag 112. The input and output controls and parameters can then be adjusted to accommodate the changes and other media or components can be added to each bag notwithstanding the designation media bag, vent bag, or cell input bag.

Those of skill in the art will further recognize that the pumps and valves in the CES serve as fluid flow controllers. In various embodiments, fluid flow controllers can be pumps, valves, or combinations thereof in any order, provided that the first fluid circulation path and second fluid circulation path are configured to circulate fluid and fluid input path(s) are configured to add fluid.

The CES can include additional components. For example, one or more pump loops (not shown) can be added at the location of peristaltic pumps on the CES. Peristaltic pumps are operably connected to the exterior of tubing, and pumps liquid through the fluid flow path by constricting the exterior of the tubing to push liquid through the tubing. The pump loops may be made of polyurethane (PU) (available as Tygothane C-210A), neoprene based material (e.g. Armapure, St. Gobain), or any other suitable material. Alternatively, a cassette for organizing the tubing lines and which may also contain tubing loops for the peristaltic pumps may also be included as part of the disposable. One or more of the components of the CES can be contained in a cassette to aid in organizing the tubing.

In various embodiments, the CES can include sensors for detecting media properties such as pH, as well as cellular metabolites such as glucose, lactate, and oxygen. The sensors can be operably associated with the CES at any location in the IC loop. Any commercially available pH, glucose, or lactate sensor can be used.

### Isolation of Naïve T-Cells

A naïve T cell (Th0 cell) is a T cell that has differentiated in bone marrow, and successfully undergone the positive and negative processes of central selection in the thymus. Among these are the naïve forms of helper T cells (CD4+) and cytotoxic T cells (CD8+).

CD45 is a protein tyrosine phosphatase regulating src-family kinases, and is expressed on all hematopoietic cells. CD45 can be expressed as one of several isoforms by alternative splicing of exons that comprise the extracellular domain. CD45RA is expressed on naïve T cells, as well as the effector cells in both CD4 and CD8. After antigen experience, central and effector memory T cells gain expression of CD45RO and lose expression of CD45RA. Thus either CD45RA or CD45RO is used to generally differentiate the naïve from memory populations.

In some embodiments, the composition disclosed herein comprise T cell populations comprising CD45A+ cells that makeup from about 0.1% to about 50% of the total T cells in the composition. In some embodiments, the composition disclosed herein comprise T cell populations comprising CD45A+ cells that makeup from about 1.0% to about 50% of the total T cells in the composition. In some embodiments, the composition disclosed herein comprise T cell populations comprising CD45A+ cells that makeup from about 10% to about 50% of the total T cells in the composition. In some embodiments, the composition disclosed herein comprise T cell populations comprising CD45A+ cells that makeup from about 20% to about 50% of the total T cells in the composition. In some embodiments, the composition disclosed herein comprise T cell populations comprising CD45A+ cells that makeup from about 30% to about 50% of the total T cells in the composition.

Differentiation between naïve and effector populations can be achieved by adding a second marker. There are several markers that have been used for this purpose and these tend to mark these populations at slightly different stages of the differentiation pathway that is thought to occur in T cells as they change from central to effector memory cells. The chemokine receptor CCR7 can be used for this discrimination, and the lymph node homing receptor CD62L is a close second choice. Naïve and central memory cells express these receptors in order to migrate to secondary lymphoid organs, while the absence of these receptors allows for effector memory and effector cells to accumulate in peripheral tissues. Other potential markers are CD27 and CD28 which are also more highly expressed by the central memory and naive populations.

Accordingly, in some embodiments, naîve T cells are CD45RA+CD45RO−CCR7+CD62L+, central memory T cells are CD45RA−CD45RO+CCR7+CD62L+, effector memory T cells are CD45RA−CD45RO+CCR7−CD62L−, and effector cells are CD45RA+CD45RO−CCR7−CD62L−.

Stimulating Naïve T Cells with Peptide-Pulsed Dendritic Cells

Prior to stimulating naïve T-cells with the dendritic cells, it may be preferable to irradiate the DCs. The DCs and naive T-cells are then co-cultured. The naïve T-cells can be co-cultured in a ratio range of DCs to T cells of about 1:5-1:50, for example, 1:5; 1:10, 1:15, 1:20, 1:25, 1:30, 1:35, 1:40, 1:45, or about 1:50. The DCs and T-cells are generally co-cultured with cytokines. In one embodiment, the cytokines are selected from a group consisting of IL-6 (100 ng/mL), IL-7 (10 ng/mL), IL-15 (5 ng/mL), IL-12 (10 ng/mL), and IL-21 (10 ng/mL).

### Second T Cell Stimulation

In general, it may be preferable to further stimulate the T-cell subpopulations with one or additional stimulation procedures. The additional stimulation can be performed with, for example, fresh DCs pulsed with the same peptides as used in the first stimulation, similarly to as described above. In one embodiment, the cytokines used during the second stimulation are selected from a group consisting of IL-7 (10 ng/mL) and IL-2 (100 U/mL).

Alternatively, peptide-pulsed PHA blasts can be used as the antigen presenting cell. The use of peptide-pulsed PHA blasts to stimulate and expand T-cells are well known in the art. Non-limiting exemplary methods can be found in Weber et al., Clin Cancer Res. 2013 Sep. 15; 19(18): 5079-5091 and Ngo et al., J Immunother. 2014 May; 37(4): 193-203, which are incorporated herein by reference. The peptide-pulsed PHA blasts can be used to expand the T-cell subpopulation in a ratio range of PHA blasts to expanded T cells of 10:1-1:10. For example, the ratio of PHA blasts to T cells can be 10:1, between 10:1 and 9:1, between 9:1 and 8:1, between 8:1 and 7:1, between 7:1 and 6:1, between 6:1 and 5:1, between 5:1 and 4:1, between 4:1 and 3:1, between 3:1 and 2:1, between 2:1 and 1:1, between 1:1 and 1:2, between 1:2 and 1:3, between 1:3 and 1:4, between 1:4 and 1:5, between 1:5 and 1:6, between 1:6 and 1:7, between 1:7 and 1:8, between 1:8 and 1:9, between 1:9 and 1:10. In general, cytokines are included in the co-culture, and are selected from the group consisting of IL-7 (10 ng/mL) and IL-2 (100 U/mL).

T cell activation and expansion is described in U.S. Provisional Application Ser. No. 62/663,239; Filed Apr. 26, 2018, incorporated by reference in its entirety herein.

### Additional T-Cell Expansion and T-Cell Subpopulation Harvest

Additional T cell stimulations may be necessary. In some embodiments, a second T cell stimulation is performed. In other embodiments, a third T cell stimulation is performed. In other further embodiments, a fourth or fifth T cell stimulation is performed.

In some embodiments, sufficient cells even for the highest cell doses required for the treatment of subjects can be prepared employing two stimulations employing methods of the present disclosure taking in the range of about 15-20 days of T cell culture, for example about 15, about 16, about 17, about 18, about 19 or about 20 days of T cell culture compared to 30 days of T cells culture standard in the art.

In some embodiments, after the first stimulation, the dendritic cells that were cryopreserved will be thawed, washed, counted, and then pulsed with peptides as described herein. Once the cells have been pulsed and the incubation period complete, the DCs will be irradiated, if necessary, and then added back to the cell expansion system. Prior to adding the DCs back to the cell expansion system, the expanded T cells will be harvested from the cell expansion system, washed, and counted. They will then be loaded back into the cell expansion system along with the dendritic cells. Alternatively, the dendritic cells will be added straight to the cell expansion system device containing the expanded T cells once the T cells were counted using the sampling coil. The stimulation ratio will be between about 1:5-1:50, for example, 1:5; 1:10, 1:15, 1:20, 1:25, 1:30, 1:35, 1:40, 1:45, or about 1:50. In certain embodiments, the stimulation ratio will be about 1:5, 1:10, 1:15, 1:20, 1:25, 1:30, 1:35 1:40, 1:45, or about 1:50. In certain embodiments, the stimulation ratio will be about 1:5. Prior to adding the DC or T cells to the cell expansion system, the cells will be re-suspended in CTL media containing IL-7 and IL-2.

In one embodiment, about 5-7 days after the first stimulation, the dendritic cells that were cryopreserved will be thawed, washed, counted, and then pulsed with peptides as described above. Once the cells have been pulsed and the incubation period complete, the DCs will be irradiated, if necessary, and then added back to the Quantum. Prior to adding the DCs back to the Quantum, the expanded T cells will be harvested from the Quantum using the Harvest task, washed, and counted. They will then be loaded back into the Quantum system along with the dendritic cells. Alternatively, the dendritic cells will be added straight to the Quantum device containing the expanded T cells once the T cells were counted using the sampling coil. The stimulation ratio will be between about 1:5-1:50, for example, 1:5; 1:10, 1:15, 1:20, 1:25, 1:30, 1:35, 1:40, 1:45, or about 1:50. In certain embodiments, the stimulation ratio will be about 1:5, 1:10, 1:15, 1:20, 1:25, 1:30, 1:35 1:40, 1:45, or about 1:50. In certain embodiments, the stimulation ratio will be about 1:5. Prior to adding the DC or T cells to the Quantum, the cells will be resuspended in CTL media containing 10 ng/mL of IL-7 and 100 U/mL of IL-2.

Following any stimulation and expansion, the T-cell subpopulations are harvested, washed, and concentrated. In one embodiment, a solution containing a final concentration of 10% dimethyl sulfoxide (DMSO), 50% human serum albumin (HSA), and 40% Hank's Balanced Salt Solution (HBSS) will then be added to the cryopreservation bag. In one embodiment, the T-cell subpopulations will be cryopreserved in liquid nitrogen.

T cell expansion may be evaluated by counting viable CD3+ cells.

Viable cells can be tested by cell staining with, for example Trypan blue (and light microscopy) or 7-amino-actinomycin D, vital dye emitting at 670 nm (or ViaProbe a commercial ready-to-use solution of 7AAD) and flow cytometry, employing a technique known to those skilled in the art. Where the stain penetrates into the cells the cells are considered not viable. Cells which do not take up dye are considered viable. An exemplary method may employ about 5μ\acute{ι} of 7AAD and about 5μ\acute{ι} of Annexin-V (a phospholipid-binding protein which binds to external phospholipid phosphatidylserine exposed during apotosis) per approximate IOOμ\acute{ι} of cells suspension. This mixture may be incubated at ambient temperature for about 15 minutes in the absence of light. The analysis may then be performed employing flow cytometry. See for example MG Wing, AMP Montgomery, S. Songsivilai and JV Watson. An Improved Method for the Detection of Cell Surface Antigens in Samples of Low Viability using Flow Cytometry. J Immunol Methods 126: 21-27 1990.

Cell expansion as employed herein refers to increasing the number of the target cells in a population of cells as a result of cell division.

### Terminal Harvest

Once the cells are ready to be used, which should coincide with the stimulation day of the ex vivo expanded antigen-specific T cells, the number of phytohemagglutinin (PHA) blasts needed will be estimated based on the number of antigen-specific T cells available in the Quantum. When using PHA blasts as antigen-presenting cells, a ratio of about 10 PHA blasts:1 antigen-specific T cell is preferred, for example a ratio of about 10 PHA blasts:1 antigen-specific T cell, about 9 PHA blasts:1 antigen-specific T cell, about 8 PHA blasts:1 antigen-specific T cell, about 7 PHA blasts:1 antigen-specific T cell, about 6 PHA blasts:1 antigen-specific T cell, about 5 PHA blasts:1 antigen-specific T cell, about 4 PHA blasts:1 antigen-specific T cell, about 3 PHA blasts:1 antigen-specific T cell, about 2 PHA blasts:1 antigen-specific T cell, about 1 PHA blasts:1 antigen-specific T cell, about 10 PHA blasts:2 antigen-specific T cell, about 9 PHA blasts:2 antigen-specific T cells, about 8 PHA blasts:2 antigen-specific T cells, about 7 PHA blasts:2 antigen-specific T cells, about 6 PHA blasts:2 antigen-specific T cells, about 5 PHA blasts:2 antigen-specific T cells, about 4 PHA blasts:2 antigen-specific T cells, about 3 PHA blasts:2 antigen-specific T cells, about 2 PHA blasts:2 antigen-specific T cells, about 1 PHA blasts:2 antigen-specific T cells, about 10 PHA blasts:3 antigen-specific T cell, about 9 PHA blasts:3 antigen-specific T cells, about 8 PHA blasts:3 antigen-specific T cells, about 7 PHA blasts:3 antigen-specific T cells, about 6 PHA blasts:3 antigen-specific T cells, about 5 PHA blasts:3 antigen-specific T cells, about 4 PHA blasts:3 antigen-specific T cells, about 3 PHA blasts:3 antigen-specific T cells, about 2 PHA blasts:3 antigen-specific T cells, about 1 PHA blasts:3 antigen-specific T cells, about 10 PHA blasts:4 antigen-specific T cell, about 9 PHA blasts:4 antigen-specific T cells, about 8 PHA blasts:4 antigen-specific T cells, about 7 PHA blasts:4 antigen-specific T cells, about 6 PHA blasts:4 antigen-specific T cells, about 5 PHA blasts:4 antigen-specific T cells, about 4 PHA blasts:4 antigen-specific T cells, about 3 PHA blasts:4 antigen-specific T cells, about 2 PHA blasts:4 antigen-specific T cells, about 1 PHA blasts:4 antigen-specific T cells, about 10 PHA blasts:5 antigen-specific T cell, about 9 PHA blasts:5 antigen-specific T cells, about 8 PHA blasts:5 antigen-specific T cells, about 7 PHA blasts:5 antigen-specific T cells, about 6 PHA blasts:5 antigen-specific T cells, about 5 PHA blasts:5 antigen-specific T cells, about 4 PHA blasts:5 antigen-specific T cells, about 3 PHA blasts:5 antigen-specific T cells, about 2 PHA blasts:5 antigen-specific T cells, about 1 PHA blasts:5 antigen-specific T cells, about 10 PHA blasts:6 antigen-specific T cell, about 9 PHA blasts:2 antigen-specific T cells, about 8 PHA blasts:2 antigen-specific T cells, about 7 PHA blasts:6 antigen-specific T cells, about 6 PHA blasts:6 antigen-specific T cells, about 5 PHA blasts:6 antigen-specific T cells, about 4 PHA blasts:6 antigen-specific T cells, about 3 PHA blasts:6 antigen-specific T cells, about 2 PHA blasts:6 antigen-specific T cells, about 1 PHA blasts:6 antigen-specific T cells, about 10 PHA blasts:7 antigen-specific T cell, about 9 PHA blasts:7 antigen-specific T cells, about 8 PHA blasts:7 antigen-specific T cells, about 7 PHA blasts:7 antigen-specific T cells, about 6 PHA blasts:7 antigen-specific T cells, about 5 PHA blasts:7 antigen-specific T cells, about 4 PHA blasts:7 antigen-specific T cells, about 3 PHA blasts:7 antigen-specific T cells, about 2 PHA blasts:7 antigen-specific T cells, about 1 PHA blasts:7 antigen-specific T cells, about 10 PHA blasts:8 antigen-specific T cell, about 9 PHA blasts:8 antigen-specific T cells, about 8 PHA blasts:8 antigen-specific T cells, about 7 PHA blasts:8 antigen-specific T cells, about 6 PHA blasts:8 antigen-specific T cells, about 5 PHA blasts:8 antigen-specific T cells, about 4 PHA blasts:8 antigen-specific T cells, about 3 PHA blasts:8 antigen-specific T cells, about 2 PHA blasts:8 antigen-specific T cells, about 1 PHA blasts:8 antigen-specific T cells, about 10 PHA blasts:9 antigen-specific T cell, about 9 PHA blasts:9 antigen-specific T cells, about 8 PHA blasts:2 antigen-specific T cells, about 7 PHA blasts:9 antigen-specific T cells, about 6 PHA blasts:9 antigen-specific T cells, about 5 PHA blasts:9 antigen-specific T cells, about 4 PHA blasts:9 antigen-specific T cells, about 3 PHA blasts:9 antigen-specific T cells, about 2 PHA blasts:9 antigen-specific T cells, about 1 PHA blasts:9 antigen-specific T cells, about 10 PHA blasts:10 antigen-specific T cell, about 9 PHA blasts:10 antigen-specific T cells, about 8 PHA blasts:10 antigen-specific T cells, about 7 PHA blasts:10 antigen-specific T cells, about 6 PHA blasts:10 antigen-specific T cells, about 5 PHA blasts:10 antigen-specific T cells, about 4 PHA blasts:10 antigen-specific T cells, about 3 PHA blasts:10 antigen-specific T cells, about 2 PHA blasts:10 antigen-specific T cells, about 1 PHA blasts:10 antigen-specific T cells. In one embodiment, the ratio is about 4 PHA blasts:1 antigen-specific T cell. The number of PHA blasts needed will be determined and about 25% to about 75%, preferably 50%, will be added to take into account cell death during irradiation. The PHA blasts will be irradiated at 75 Gy, washed (if applicable), and then resuspended in CTL media along with 100 U/mL of IL-2. As above, the cells will be fed on day 3-4 or the media containing IL-2 will be perfused continuously. After 5-7 days, the cells will be harvested using the Harvest task on the Quantum. Once harvested, cells will be washed and concentrated on the Lovo or similar device; a solution containing a final concentration of 10% DMSO, 50% HSA, and 40% plasmalyte (or similar) will then be added to the cryopreservation bag. The bag will be transferred to a control rate freezer where the cells will be cryopreserved.

### Compositions and Pharmaceutical Compositions

The present disclosure also extends to compositions comprising the T cell populations as described herein. These compositions may comprise a diluent, carrier, stabilizer, surfactant, pH adjustment or any other pharmaceutically acceptable excipient added to the cell population after the main process steps. An excipient will generally have a function of stabilizing the formulation, prolonging half-life, rendering the composition more compatible with the in vivo system of the patient or the like.

In one embodiment a protein stabilizing agent is added to the cell culture after manufacturing, for example albumin, in particular human serum album, which may act as a stabilizing agent. The amounts albumin employed in the formulation may be about 10% to about 50% w/w, such as about 12.5% w/w.

In one embodiment the formulation also contains a cryopreservative, such as DMSO. The quantity of DMSO is generally about 20% or less such as about 12% in particular about 10% w/w.

In embodiment the process of the present invention comprises the further step of preparing a pharmaceutical formulation by adding a pharmaceutically acceptable excipient, in particular an excipient as described herein, for example diluent, stabilizer and/or preservative.

Excipient as employed herein is a generic term to cover all ingredients added to the T cell population that do not have a biological or physiological function.

Once the final formulation has been prepared it will be filled into a suitable container, for example an infusion bag or cryovial.

In one embodiment the process according to the present disclosure comprises the further step of filling the T cell population or pharmaceutical formulation thereof into a suitable container, such as an infusion bag and sealing the same.

In one embodiment the container filled with the T cell population of the present disclosure or a pharmaceutical composition comprising the same is frozen for storage and transport, for example is store at about −135° C., for example in the vapor phase of liquid nitrogen.

In one embodiment the process of the present disclosure comprises the further step of freezing the T cell population of the present disclosure or a pharmaceutical composition comprising the same. In one embodiment the “product” is frozen by a controlled rate freezing process, for example reducing the temperature by 1° C. per minute to ensure the crystals formed are small and do not disrupt the cell structure. This process may be continued until the sample has reached about −100° C.

A product according to the present disclosure is intended to refer to a cultured cell population of the present disclosure or a pharmaceutical composition comprising the same.

In one embodiment the product is transferred, shipped, transported in a frozen form to the patient's location.

In one embodiment the product according to the present disclosure is provided in a form suitable for parenteral administration, for example infusion, slow injection or bolus injection. In one embodiment the formulation is provided in a form suitable for intravenous infusion.

In one aspect the present disclosure provides a method of transporting a product according to the present disclosure, from the place of manufacture, or a convenient collection point to the vicinity of the intended patient, for example where the T cell product is stored below 0° C., such as −135° C. during transit.

In one embodiment the temperature fluctuations of the T cell product are monitored during storage and/or transport.

### Treatment Methods

In one embodiment there is provided a product of the present disclosure for use in treatment, for example, in the treatment of hematological and solid tumors and in the treatment of non-cancer disorders, such as autoimmune diseases and disorders.

In one embodiment the treatment is of an immunosuppressed patient.

In one embodiment, the patient is not immune-compromised.

In one embodiment there is a provided a method of treating a patient with a product according to the present disclosure comprising the step of administering a therapeutically effective amount of product defined herein.

Therapeutically effective amount, does not necessarily mean an amount that is immediately therapeutically effective but includes a dose which is capable for expansion in vivo (after administration) to provide a therapeutic effect.

Thus there is provided a method of administering to a patient a sub-therapeutic dose but nonetheless becoming a therapeutically effective amount after expansion of T cells in vivo to provide the desired therapeutic effect, for example. In some embodiments, a sub-therapeutic dose is an amount that is less than the therapeutically effective amount.

Hematological and Solid Tumors Targeted for Treatment

The T-cells described herein can be used to treat a patient with a solid or hematological malignancy.

Lymphoid neoplasms are broadly categorized into precursor lymphoid neoplasms and mature T-cell, B-cell or natural killer cell (NK) neoplasms. Chronic leukemias are those likely to exhibit primary manifestations in blood and bone marrow, whereas lymphomas are typically found in extramedullary sites, with secondary events in the blood or bone. Over 79,000 new cases of lymphoma were estimated in 2013. Lymphoma is a cancer of lymphocytes, which are a type of white blood cell. Lymphomas are categorized as Hodgkin's or non-Hodgkin's. Over 48,000 new cases of leukemias were expected in 2013.

In one embodiment, the disease or disorder is a hematological malignancy selected from a group consisting of leukemia, lymphoma and multiple myeloma.

In one embodiment, the methods described herein can be used to treat a leukemia. For example, the patient such as a human may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In one embodiment, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In a particular embodiment, the hematological malignancy is a lymphoma or lymphocytic or myelocytic proliferation disorder or abnormality. In one embodiment, the lymphoma is a non-Hodgkin's lymphoma. In one embodiment, the lymphoma is a Hodgkin's lymphoma. In one embodiment, the hematological malignancy is a relapsed or refractory leukemia, lymphoma, or myeloma.

In some aspects, the methods described herein can be used to treat a patient such as a human, with a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the methods described herein can be used to treat a patient, such as a human, with a Hodgkin's Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, the methods described herein can be used to treat a patient, for example a human, with specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

Abnormal proliferation of T-cells, B-cells, and/or NK-cells can result in a wide range of cancers. A host, for example a human, afflicted with any of these disorders can be treated with an effective amount of the TAA-L composition as described herein to achieve a decrease in symptoms (a palliative agent) or a decrease in the underlying disease (a disease modifying agent).

In some embodiments, the T-cells and T-cell compositions described herein can be used to treat a hematological malignancy, for example but not limited to T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some aspects, the tumor is a solid tumor. In one embodiment, the solid tumor is Wilms Tumor. In one embodiment, the solid tumor is osteosarcoma. In one embodiment, the solid tumor is Ewing's sarcoma. In one embodiment, the solid tumor is neuroblastoma. In one embodiment, the solid tumor is soft tissue sarcoma. In one embodiment, the solid tumor is rhabdomyosarcoma. In one embodiment, the solid tumor is glioma. In one embodiment, the solid tumor is germ cell cancer. In one embodiment, the solid tumor is breast cancer. In one embodiment, the solid tumor is lung cancer. In one embodiment the solid tumor is ovarian cancer. In one embodiment, the solid tumor is renal cell carcinoma. In one embodiment, the solid tumor is colon cancer. In one embodiment, the solid tumor is melanoma. In one embodiment, the solid tumor is a relapsed or refractory solid tumor.

Non-limiting examples of tumors that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast, triple negative breast cancer, HER2-negative breast cancer, HER2-positive breast cancer, male breast cancer, late-line metastatic breast cancer, progesterone receptor-negative breast cancer, progesterone receptor-positive breast cancer, recurrent breast cancer), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

### Non-Cancer Disorders

The T-cells and T-cell compositions described herein can be used to treat a patient with a non-cancer disorder. In one embodiment, the disease or disorder is an autoimmune disease.

In one embodiment, the T-cells and T-cell compositions can be used to treat a patient with an autoimmune disease. Non-limiting examples of autoimmune diseases that can be treated with HSCT include, but are not limited to, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diamond-Blackfan anemia, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Hemophagocytic lymphohistiocytosis (HLH), Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the disclosure, and, as such, may be considered in making and practicing the invention. Particularly useful techniques for particular embodiments will be discussed in the sections that follow.

### Methods of Cell Isolation and Culture

The disclosure also relates to a method of culturing lymphocytes in any one of the tissue culture systems disclosed herein. In some embodiments, the disclosure provides an extended periodic harvest method which offers the advantage of maintaining a continuous cell culture while maintaining purity of disclosed T-cell populations. The invention provides a method for extended periodic harvest comprising establishing a cell culture by inoculating a bioreactor with PBMCs cells comprising a naïve T cell population or a CD45RA+ T-cell population, maintaining the cell culture by perfusing fresh cell culture medium onto at least one cell reactor surface, optionally passing the cell culture through at least one filter; optionally exposing the cell reactor surface to one or more cytokines, and then harvesting the cells after a time period sufficient to proliferate the CD45RA+ T cells to a first predetermined parameter is reached, at which time a harvest permeate is collected for a predetermined time. In some embodiments, the methods of isolating or culturing disclosed T cells populations (such as CD45RA+ T cells) comprises exposing the T cell populations to adherent or non-adherent antigen presenting cells, such as dendritic cells. In some embodiments, the methods provide for stimulating any one or plurality of antigen presenting cells with tumor antigens prior to exposure to the T cell populations. The methods disclosed can be performed in a closed system such as the system disclosed in FIG. 5, whereby the cell culture unit (502) can be accessed in a sterile fashion by one or a plurality of ports in fluid communication with a cell culture chamber. Introduction of media, cytokines, chemokines or other cells to the T cell population can occur so that the environment for T cell stimulation of naïve T cells can occur prior to harvest.

In some embodiments, the predetermined parameters may be reached by achieving some desired characteristic, attribute or performance milestone of the cell culture; such as viable cell density, packed cell volume or titer or a time point sufficient to stimulate the CD45RA+ cells with one or a plurality of antigens disclosed herein. In one embodiment, the predetermined parameter may be reached when the viable cell density is greater than or equal to 1×10⁶ viable cells/ml. In one embodiment, predetermined parameter may be reached when the viable cell density is at least 20×10⁶ viable cells/ml to 30×10⁶ viable cells/ml. In one embodiment, predetermined parameter may be reached when the packed cell volume is less than or equal to 35%. In one embodiment, predetermined parameter may be reached when the packed cell volume is less than or equal to 30%. In some embodiments, the predetermined parameter is a time period of from about 1 to 10 days of expansion after any one or more of the steps disclosed herein. In some embodiments, the predetermined parameter is a time period of from about 2 to 9 days of expansion after any one or more of the steps disclosed herein. In some embodiments, the predetermined parameter is a time period of from about 3 to 7 days of expansion after any one or more of the steps disclosed herein. In some embodiments, the predetermined parameter is a time period of from about 3 to 7 days of expansion after exposure of CD45RA+ T-cells with one or more sets of dendritic cells or antigen presenting cells. In some embodiments, the dendritic cells or antigen presenting cells are stimulated with exposure to WT1, PRAME and/or survivin before exposure to the population of cultured CD45A+ T-cells. Some methods of the disclosure relate to methods of isolating naive T cells targeting a tumor antigen comprising harvesting the naïve T cells after expanding the cell to a predetermined parameter of culture that is, in some embodiments, achieving a particular cell density after expansion for about 2 to about 9 days.

In one embodiment, any of the methods of harvesting primed T cell population or culturing T cell populations disclosed herein comprise a step of perfusing the cell culture unit continuously. In one embodiment the rate of perfusion is constant. In one embodiment the perfusing is performed at a rate of less than or equal to 1.0 working volume per day. In one embodiment the perfusing is accomplished by a peristaltic pump, a double diaphragm pump, a low shear pump or alternating tangential flow. In a related embodiment the perfusing is accomplished by alternating tangential flow.

In one embodiment the method above further comprises subjecting the cell culture to a temperature shift wherein the cells are cultured a) at first temperature for a first period of time and b) at second temperature for a second period of time. In a related embodiment the temperature shift occurs at the transition between the stimulation phase and expansion phase. In a related embodiment the temperature shift occurs during the expansion phase. In a related embodiment the temperature shift is in response to a predetermined parameter. In a related embodiment the temperature shift is in response to a predetermined parameter wherein achieving the predetermined parameter is determined using a capacitance based biomass probe sampling cell culture media sterilely taken from the cell culture unit.

All of the references, patent applications, GenBank Accession numbers or other documents listed in this application and the Examples section are herein incorporated by reference in their entireties.

### EXAMPLES

### Example 1

### Testing the Cell Expansion System

The Quantum cell expansion system is an automated system with 2.1 m² of surface area, which is the equivalent of about 4 CF10s or 120 T-175 flasks. The quantum provides optimal exchange of gas and nutrients and allows the user to set variable perfusion rates. The Quantum has rapid harvest time: instead of taking 8 hours and four people to harvest 256 flasks, 500 million cells can be harvested in 30 minutes.

To test the quantum, the following protocol is set us: prime one Quantum a day before the arrival of the bone marrow. Once the bone marrow arrives, wash the bioreactor, condition the media, and then load the cells through a 200 micron filter. The mononuclear cells are not isolated beforehand. The system is monitored daily and the feed rate is increased as needed. Anticipating a harvest on day 10, Quantum #2 is coated on day 9 because the bioreactor in Quantum #1 cannot be coated if the cells are still growing in it. On day 10 the cells are harvested from Quantum #1, counted them, and sent for phenotyping and functional analyses. The cells are then loaded in Quantum #2 at 10-40 million cells/bioreactor.

### Example 2 Isolating and Expanding Naïve T Cells Targeting TAAs in a Closed System

During the manufacture of T cells specific for Tumor Associated Antigens (TAA) or virus-specific T cells where the donor is seronegative (such as cord blood or adult serongegative donors), the expanded T cell product will be derived from the naïve T cell population instead of the memory T cell population, which has been the source of T cells in many other cellular therapy protocols. Therefore, it is anticipated that by selecting for the CD45RA+ T cells, the population that will respond to stimulation will be enriched, leading to a superior expansion and final therapeutic product.

Described in this Example is the isolation of naïve cells, their expansion, and final harvest in a way that is closed and semi-automated.

### Steps:

Starting Product Collection

The starting product is an apheresis mononuclear cell product collected from a non-mobilized donor. The product will be collected from a healthy donor or patient and apheresed using a collection machine such as the Spectra Optia or similar.

Processing the Apheresis Product

Using the Elutra device by Terumo, leukapheresis products will be processed according to the manufacturer's recommendations. The device uses count-flow centrifugation with a fixed rotor speed of 2400 RPM. It also uses a computer to adjust the medium flow rate. The Elutra is capable of separating leukocytes using their unique physical characteristics. It is capable of separating the starting product into separate bags with platelets, red blood cells, lymphocytes, and monocytes. The monocyte fraction will be used as detailed below. The lymphocyte fraction will be cryopreserved until the cell selection step. Small aliquots of the lymphocyte fraction (˜5×10⁷ cells) will be cryopreserved separately for Phytohemagglutinin (PHA) Blast expansion, if needed.

Generating Dendritic Cells

To generate dendritic cells from the monocyte fraction bag, the monocyte fraction will be plated into a closed system bioreactor such as the Quantum Cell Expansion System. The Quantum has a surface area of 2.1 m², so approximately 7×10⁹ cells from the monocyte fraction will be added via the Cell Inlet bag on the Intracapillary (IC) line of the Quantum cell expansion system to yield a cell density of 3.3×10⁵ cells/cm². The cells will be allowed to adhere for 2-4 hours at which point 1,000 U/mL of IL-4 and 800 U/mL GM-CSF will be added to the Quantum via the reagent bag of the IC line. Cells will be re-fed after 1-2 days with the same concentration of GM-CSF and IL-4. On day 2-5 (ideally day 2), the cells will be matured using a cytokine cocktail including LPS (30 ng/mL), IL-4 (1,000 U/mL), GM-CSF (800 U/mL), TNF-Alpha (10 ng/mL), IL-6 (100 ng/mL), and IL-1beta (10 ng/mL). One-to-two days after maturation, cells will be harvested from the Quantum. To harvest the cells, media will be added at a high rate into the collection back; this will collect all non-adherent cells. To then harvest the adherent cells, we will use the Harvest task that is pre-loaded on the device. The cells will be incubated with TrypLE select or a similar dissociation reagent for 10-15 minutes at which point the Release Cells task will harvest all cells into the Harvest bag. If necessary, a new bag can be loaded onto the harvest line to accommodate additional volume or washes to collect all cells.

To volume reduce the media, rid of the cells of unwanted media and growth factors, and concentrate the cells, the cells will then be processed on the Lovo automated cell processing system or a similar device like the Sepax; alternatively, the bag can be centrifuged and the supernatant expressed off into another bag. Once the volume is reduced to ˜100 mL (range 10-250 mL), ½-¾ of the cells will be removed and cryopreserved to be used for the second stimulation. To the second fraction, 100 ng of each peptide (or 100 ng of a peptide mixture) will be added per 10 million dendritic cells. The pepmixes will be added using a luer lock or port on the bag. The bag will be mixed periodically and incubated with the peptides for 30 minutes to 2 hours. Once the incubation period is complete, the cells will then be re-loaded into the Quantum Cell Expansion System, loading the dendritic cells at a 1:5-1:50 ratio of dendritic cells to lymphocytes.

Alternative

Alternatively, half the apheresis product will be loaded at initiation and the remainder frozen for the second stimulation. The dendritic cells will be left in the Quantum bioreactor after maturation and the peptides will be added directly to the IC line using the reagent bag. An approximate cell count will be obtained using the sampling coil.

Naïve T Cell Selection of Lymphocytes and T Cell: DC Co-Culture

Around the same time or during the time that the dendritic cells are being harvested, the lymphocytes from the non-adherent fraction will be thawed, washed using the lovo or similar device, and resuspended in CliniMACS buffer containing 0.5% human serum albumin. A small aliquot of cells will be removed for counting and quality control, including flow cytometry. To select for CD45RA+ cells, the cells will be labeled using 1 vial of CD45RA microbeads from Miltenyi Biotec per 1×10¹¹ cells after 5-30 minutes of incubation with 100 mL of CliniMACS buffer and approximately 3 mL of 10% human WIG, 10 ug/mL DNAase I, and 200 mg/mL of magnesium chloride. The cells will be incubated with this mixture for around 30 minutes. After 30 minutes, cells will be washed sufficiently using the Lovo (or twice if using centrifugation) and resuspended in 20 mL of MACS buffer. The bag will then be set up on the CLINIMACS Plus device or the Prodigy using the LS or appropriate tubing set and the selection program will be run according to manufacturer's recommendations. After the program is completed, a sample will be removed from QC (including post-selection CD45RA analysis) and cell count, washed using the Lovo or similar, and resuspended in “CTL Media” consisting of 44.5% EHAA Click's, 44.5% Advanced RPMI, 10% Human Serum, and 1% GlutaMAX. Cell counts of the selected CD45RA+ T cells will be adjusted based on the ratio of T cells to dendritic cells, which were isolated as above. The ideal ratio of DCs to lymphocytes is 1:5 with a range of 1:5-1:50 being acceptable. Dendritic cells may also be irradiated at 25 Gy prior to mixing with T cells, if necessary. Before adding the dendritic cells (or T cells in the alternative DC manufacturing protocol) to the bioreactor, the T cells will be resuspended in T cell media (defined above) as well as the cytokines IL-6 (100 ng/mL), IL-7 (10 ng/mL), IL-15 (5 ng/mL), IL-12 (10 ng/mL). Alternatively, T cells may be expanded with the dendritic cells in the Prodigy.

### 2^{nd} T Cell Stimulation

5-7 days after the first stimulation, the dendritic cells that were cryopreserved will be thawed, washed, counted, and then pulsed with peptides as described above. Once the cells have been pulsed and the incubation period complete, the DCs will be irradiated, if necessary, and then added back to the Quantum. Prior to adding the DCs back to the Quantum, the expanded T cells will be harvested from the Quantum using the Harvest task, washed, and counted. They will then be loaded back into the Quantum system along with the dendritic cells. Alternatively, the dendritic cells will be added straight to the Quantum device containing the expanded T cells once the T cells were counted using the sampling coil. The ideal stimulation ratio will be 1:5 T cells to DC with a range of 1:5 to 1:50. Prior to adding the DC or T cells to the Quantum, the cells will be resuspended in CTL media containing 10 ng/mL of IL-7 and 100 U/mL of IL-2.

Further T Cell Expansion and Feeding and Terminal Harvest

T cells will be fed with 100 U/mL of IL-2 on day 3-4, or they can be fed continuously via perfusion of the media containing IL-2. After 5-7 days of expansion, the expanded T cells will be counted; if sufficient T cells are available then the cells will be harvested using the Harvest function, washed with the lovo, and then cryopreserved in bags containing 40% plasmalyte (or similar), 50% Human Serum Albumin (HSA), and 10% DMSO.

If insufficient cells are available after 5-7 days, a third stimulation will be performed. On day 7 post initiation, a separate aliquot of lymphocytes will be thawed, washed, and added to a cell expansion bag containing CTL media and 5 ug/mL of PHA. Feed the cells with 100 U/mL of IL-2 every two days and harvest the T cells—to be used as antigen-presenting cells—after 5-7 days.

Once the cells are ready to be used, which should coincide with the stimulation day of the ex vivo expanded antigen-specific T cells, the number of PHA blasts needed will be estimated based on the number of antigen-specific T cells available in the Quantum. When using PHA blasts as antigen-presenting cells, a ratio of 4 PHA blasts:1 antigen-specific T cell is optimal, with a range of 10:1-1:10. The number of PHA blasts needed will be determined and ˜50% will be added to take into account cell death during irradiation. The PHA blasts will be irradiated at 75 Gy, washed (if applicable), and then resuspended in CTL media along with 100 U/mL of IL-2. As above, the cells will be fed on day 3-4 or the media containing IL-2 will be perfused continuously. After 5-7 days, the cells will be harvested using the Harvest task on the Quantum. Once harvested, cells will be washed and concentrated on the Lovo or similar device; a solution containing a final concentration of 10% DMSO, 50% HSA, and 40% plasmalyte (or similar) will then be added to the cryopreservation bag. The bag will be transferred to a control rate freezer where the cells will be cryopreserved.

FIG. 1 depicts a flowchart that describes the simple method that is to performed in a closed system. A closed system for purposes of the disclosure may be one that have more than one module or component but each module or component is sealed from the outside environment such that sterile cellular product may not be exposed to the outside environment. In some embodiments, the methods may be performed under good clinical manufacturing protocol such that the harvested cells, either in suspension or adherent and removed, can be used for administration to a subject, such as a human patient. It is understood that the system may have several sealable or resealable outlets or inlets, covered for instance by Luer lock, such that syringes, cannulas or contents of a similarly sealed compartments may be accessible to the system via simple fluid connection.

The disclosure relates as depicted in FIG. 1 to a method of harvesting and/or freezing cells passed through the method steps or harvesting and administering the cells stimulated in the system. Minimally, cell samples, in some embodiments, from a subject must be separated for cell type by an apheresis step 101. In some embodiments, a sample is separated into a PBMC cell fraction. CD45A+ cells may be further selected and isolated before being cultured 102. In some embodiments, the cells are free or substantially free of memory T cells. From the same separated fractions of cells, dendritic cells are selected and isolated 103.

The isolated cell identified above may then be co-cultured 104 in one or more cell reactors whereby, in some embodiments, one fraction of cells is adherent to the cell reactor surface and one fraction of cells are in suspension. While the cells co-culture in contact with one another, the cells are exposed to a composition of antigens 105. In some embodiments, the composition of antigens is a PepMix cocktail of various antigens, one or more tumor associated antigens, such as those disclosed herein and/or one or more viral antigens, such as those described herein. This step may be repeated once, twice, thrice or more until a number of CD45A+ T cells are sufficiently stimulated to associate with a cell expressing the antigen or antigens. The T cells are allowed to expand in culture 106 and then the cells may be harvested 107 and used for either administration to a patient comprising a cell expressing one or more of the antigens 108 or frozen into aliquots for later use 109. This process is novel because it is a closed process, it is capable of yielding several million cells in the range of 1×10⁹ or more in a large batch fashion per run and the resultant cells can be frozen down in aliquots according to the antigen or antigens that were exposed to the cells. Another significant advantage is that unlike several of the similar methods performed which can yield similarly stimulated T cells (without the same phenotype) in 30 or more days, this process can be performed in about 12 to about 16 days to yield T cells primed to attack cells bearing one or a plurality of antigens. In some embodiments, the steps may be performed in 12, 13, 14, 15, 16, 17, 18, or 19 days. In some embodiments, the cells may be stimulated as many as three times before being harvested.

FIG. 2 shows the Quantum bioreactor (TerumoBCT). The bioreactor is constructed of 10,000 hollow fibers that total about 2.1 meters squared of surface area. Inside the hollow fibers is the intracapillary (IC) space where cells are fed with media. Between hollow fibers is the extracapillary (EC) space which has many functions, including ultrafiltration where we can feed from the EC sign at a rate that causes non-MSC cells to detach and wash away into the waste bag.

FIG. 3 shows the components of the Quantum cell culture device: the expansion set. In the top right corner are the inlet and outlet lines, that include lines for reagents such as TrypLE select, a wash line for PBS, a cell line, and a harvest line. There is also an IC media line for feeding cells from the intracapillary space and an EC media line for feeding from the extra capillary space.

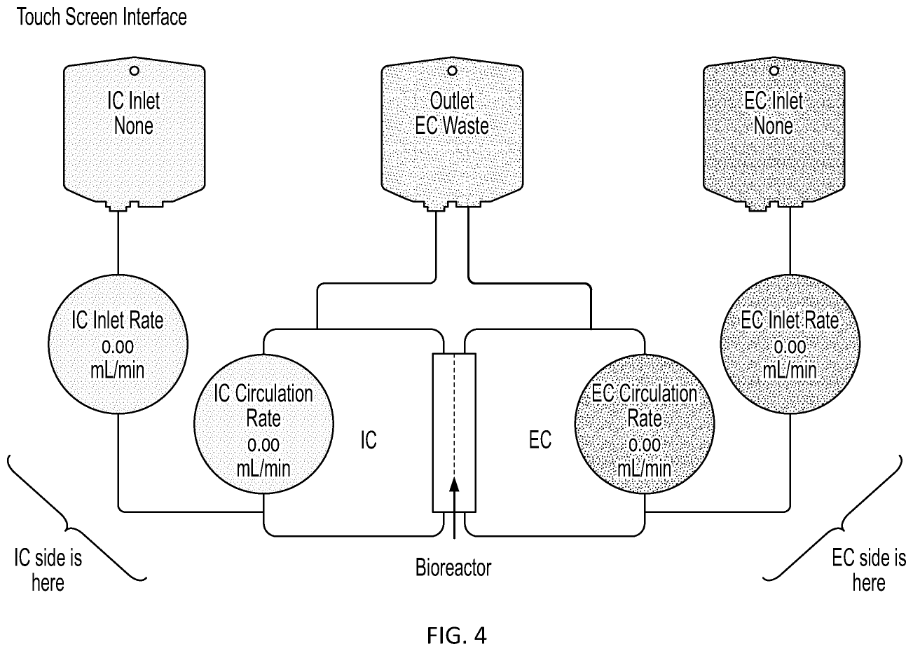

FIG. 4 shows a schematic of the touch-screen interface. There are two media lines, IC and EC. Each has its own designated inlet rate and circulation rate. Depending on the growth of the cells, we can adjust the inlet rate to feed the cells more often. One advantage of having the IC line and the EC line is that two media bags can be hooked up, and a task can be set up that will change the feeding of one bag to the other bag once the first bag is empty.

Referring to FIG. 5, in one embodiment, the antigen-specific T-cells are stimulated and expanded in a cell culture unit 502, e.g., a Quantum Cell Expansion System or other CESs as discussed elsewhere herein. T-cells can be added to cell stimulation and expansion system 502 through any method, including injection, fluid flow, etc. After T-cell stimulation and expansion as discussed elsewhere herein, the stimulated and expanded T-cells can be removed from the cell culture unit 502 through any method, including vacuum, fluid flow, etc.

In some embodiments, the cell culture unit 502 is connected to a harvesting compartment 503 for collection of stimulated and expanded T-cells. In such embodiments, the cell culture unit 502 comprises a cell stimulation and expansion system outlet port 512 that is fluidly associated via tubing with a cell harvest bag inlet port 513 into the harvesting compartment 503. Fluid flow between the cell culture unit 502 and the harvesting compartment 503 can be accomplished, for example, by a pump (e.g., mechanical or gravity-driven).

In some embodiments, an apheresis unit 501 is used. An apheresis system, as discussed elsewhere herein, generally includes a blood component separation device. T-cells separated from other blood components in the apheresis unit 501 are directed to the cell culture unit 502 through a connection tube that has an apheresis system outlet port 510 and a cell stimulation and expansion system inlet port 511. Fluid flow between the apheresis unit 501 and the cell culture unit 502 can be accomplished, for example, by a pump (e.g., mechanical or gravity-driven).

In some embodiments, cell stimulation and expansion system inlet port 511 and cell stimulation and expansion system outlet port 512 are the same and the connection tube can be used to connect either the apheresis unit 501 or the harvesting compartment 503, with fluid flow be properly routed (either into the cell culture unit 502 if from apheresis system 501 or out of cell culture unit 502 if to the cell harvest bag 503), e.g., through use of a pump. In other embodiments, cell stimulation and expansion system inlet port 511 and cell stimulation and expansion system outlet port 512 are different, with cell stimulation and expansion system inlet port 511 providing only input from apheresis unit 501 into the cell culture unit 502, and cell stimulation and expansion system outlet port 512 providing only output from the cell culture unit 502 to the harvesting compartment 503.

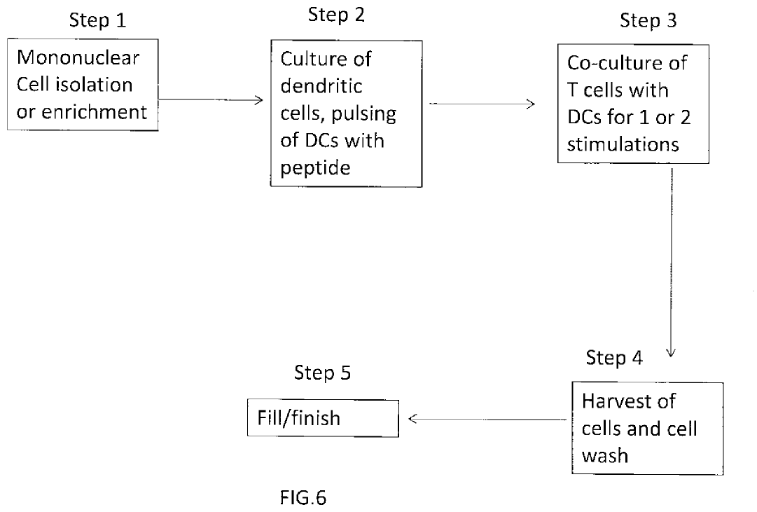

Referring to FIG. 6, in one embodiment, an alternative method to the isolation and culturing of naive T cells is depicted in which the T cells may be used for therapeutic use after stimulation or for storing until a subject in need of the T cells is identified, after which the T cells can be thawed and administered to a subject in a therapeutically effective amount. After a step of drawing blood from a donor (in some embodiments, a healthy donor of lymphocytes), Step 1 of the method comprises enrichment or isolation of PBMCs. The step comprises elimination of red blood cells, typically performed through apheresis device disclosed herein. Optionally, the step also comprises selection of cell CD14+ cells to make a culture of dendritic cells, CD3+ cells to enrich for T cells and/or selection of CD4+ or CD8+ to select specific subpopulations of T. Any Elutra device, Prodicgy device, CliniMacs device or Sepax device may be used to accomplish selection or enrichment of lymphocyte populations in culture. In some embodiments, primary samples of healthy subject sera are isolated and placed over the devices comprising an appropriate antibody to select for subpopulations of cells expressing CD14, CD3, CD4 and/or CD8. In some embodiments, the method further comprises culturing adherent dendritic cells in any tissue culture system disclosed herein and exposing the dendritic cells to one or a plurality of tumor cell antigens. In some embodiments, the tumore cell antigens are those disclosed herein and may comprise WT1 or functional epitopes thereof, and/or survivin or functional epitopes thereof and/or PRAME or functional epitopes thereof. In some embodiments, the antigens against which the dendritic cells are exposed are any one or combination of antigens disclosed in the PCT Application entitled, “IMPROVED TARGETED T-CELL THERAPY” which is filed on May 20, 2019 and claims priority to U.S. Application No. 62/673,745, both of which are incorporated by reference in their entireties. Rather than exposing the dendritic cells to an enzyme that may release the cells from any or or plurality of cell reactor surfaces, in some embodiments, the methods disclosed herein further comprise a step of introducing or co-culturing the CD45A+ T cells into the dendritic cell culture for a time period sufficient to induce an antigen-specific T-cell after exposure to the dendritic cell culture. In some embodiments, the methods also further comprise optional steps of perfusion of, filtering of or changing of the media, addition of cytokines, and/or addition of isolated antigen presenting cells other than the dendritic cell culture for stimulation of the T cell populations for proliferation. In some embodiments, the cell culture unit comprises a gasket, port and/or valve capable of sampling the cell culture medium in the closed system while also preserving the sterility of the closed system. Steps of the methods disclosed herein may also include testing the tissue cell media for quantification or detection of metabolites, secreted molecules, or non-adherent cells present in the culture system.

1-11. (canceled)

12. A method of expanding CD45A+ T-cells from a subject comprising:
(a) culturing one or a plurality of CD45A+ T-cells in a cell culture system comprising:
one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet;
a gas transfer module in operable connection to the one or plurality of cell reactor surfaces; and
a first gas inlet in operable connection to the gas transfer module; and
(b) allowing the CD45A+ T-cells to grow in the first compartment for a time period sufficient to proliferate.

13. The method of claim 12 further comprising introducing CD45A+ T-cells into the first compartment of the system.

14. The method of claim 12, wherein the CD45A+ T-cells are allowed to grow for a time period sufficient to proliferate into a total cell number of from about 1×10⁹ to about 1×10¹² cells.

15. The method of any of claim 12, wherein the step of culturing comprises co-culturing the CD45A+ T-cells with one or a plurality of dendritic cells.

16. The method of claim 12 further comprising a step of allowing one or plurality of dendritic cells presenting at least one antigen to contact one or plurality of CD45A+ T-cells for a period of time sufficient to stimulate a T-cell response against the at least one antigen.

17. The method of claim 12, wherein the dendritic cells and the CD45A+ T-cells are from a subject.

18. A method of isolating antigen-stimulated CD45A+ T-cells comprising:
(a) culturing one or a plurality of CD45A+ T-cells in a cell culture system comprising;
one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet;
a gas transfer module in operable connection to the one or plurality of cell reactor surfaces; and
a first gas inlet in operable connection to the gas transfer module;
(b) allowing the CD45A+ T-cells to grow within the first compartment for a time period sufficient to proliferate;
(c) allowing one or plurality of dendritic cells presenting at least one antigen to contact one or plurality of CD45A+ T-cells for a period of time sufficient to stimulate a T-cell response against the at least one antigen; and
(d) harvesting the one or plurality of CD45A+ T-cells in a closed system.

19. The method of claim 18 further comprising repeating step (c) one or more times before performing step (d).

20. The method of claim 18, wherein the total time to perform all of the steps is less than 20 days.

21. The method of claim 18, wherein the total time to perform all of the steps is from about 12 to about 16 days.

22. The method of claim 18, wherein the antigen-stimulated CD45A+ T cells are cultured in media comprising about 44.5% EHAA Click's, about 44.5% Advanced RPMI, about 10% Human Serum, and about 1% GlutaMAX.

23-27. (canceled)

28. A method of creating a library of antigen-stimulated T cells comprising:
(a) culturing one or a plurality of T-cells in a cell culture system comprising:
one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet;
a gas transfer module in operable connection to the one or plurality of cell reactor surfaces; and
a first gas inlet in operable connection to the gas transfer module;
(b) allowing the T-cells to grow within the first compartment for a time period sufficient to proliferate;
(c) allowing one or plurality of dendritic cells presenting at least one antigen to contact one or plurality of T-cells for a period of time sufficient to stimulate a T-cell response against the at least one antigen; and
(d) harvesting the one or plurality of T-cells in a closed system.

29. The method of claim 28 further comprising a step of sorting cells into aliquots based upon the antigens against which the T-cell response is raised.

30. The method of claim 28, wherein the total time to perform all of the steps is less than 20 days.

31. The method of claim 28, wherein the total time to perform all of the steps is from about 12 to about 16 days.

32. The method of claim 12, wherein the T cells are free of or substantially free of memory T cells.

33-39. (canceled)

40. The method of claim 18, wherein the T cells are free of or substantially free of memory T cells.

41. The method of claim 28, wherein the T cells are free of or substantially free of memory T cells.
